(19) United States
(12) Patent Application Publication
(10) Pub. No.: US 2008/0021038 A1
(43) Pub. Date: Jan. 24, 2008

(54) Novel Piperidine/8-Azabicyclo [3.2.1.] Octan Derivatives As Modulators Of Chemokine Receptor Ccr5

(76) Inventors: Howard Tucker, Macclesfield (GB); Alan Faull, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 11/628,808

(22) PCT Filed: Jun. 20, 2005
(86) PCT No.: PCT/SE05/00953

§ 371 Date: Dec. 7, 2006

(30) Foreign Application Priority Data
Jun. 24, 2004 (SE) 0401656-4

(51) Int. Cl. A61K 31/496; A61K 31/445; A61P 37/00; C07D 221/00; C07D 221/02; C07D 295/00

(52) U.S. Cl. 514/253.01; 514/299; 514/316; 514/317; 514/326; 544/360; 546/183; 546/186; 546/210; 546/236

(57) ABSTRACT

Compounds of formula (I) wherein neither R⁴ nor R⁵ is hydrogen; compositions comprising them, processes for preparing them and their use in medical therapy (for example modulating CCR5 receptor activity in a warm blooded animal).

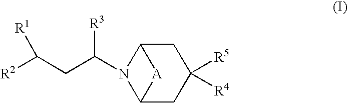

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in WO03/030898.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C, or α) and Cys-Cys (C-C, or β) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1,β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I): 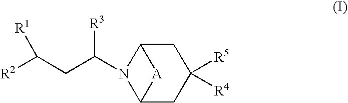
wherein:
A is absent or it is CH₂CH₂;
R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, heterocyclyl, aryl or heteroaryl;
R¹⁴, R¹⁷, R¹⁹, R²⁰ and R²² are hydrogen or C₁₋₆ alkyl;
R¹⁵, R¹⁶, R¹⁸, R²¹ and R²³ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₚ(C₁₋₆ alkyl), halo or C₁₋₄ alkyl); or R¹⁵, R¹⁶, R¹⁸ and R²¹ can also be hydrogen; or R¹⁴ and R¹⁵, and/or R²⁰ and R²¹ may join to form a 4-, 5- or 6-membered ring which optionally includes a nitrogen, oxygen or sulphur atom, said ring being optionally substituted by halo, C₁₋₆ alkyl, S(O)₁(C₁₋₆ alkyl) or C(O)(C₁₋₆ alkyl);
R² is phenyl or heteroaryl, either of which is optionally substituted by halo, C₁₋₄ alkyl, C₁₋₄ alkoxy, cyano or CF₃;
R³ is hydrogen or C₁₋₄ alkyl;
R⁴ is halo, hydroxy, cyano, C₁₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);
R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) or C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl (optionally substituted by halogen, C₁₋₄ alkoxy or OH), H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring; the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro};
X is O, S(O)ₚ, S(O)₂NR⁸ or NR⁸S(O)₂;
m and n are 1, 2 or 3;
R⁶ is hydrogen, methyl, ethyl, allyl or cyclopropyl;
R⁷ is phenyl, heteroaryl, phenylNR¹¹, heteroarylNR¹¹, phenyl(C₁₋₂)alkyl, heteroaryl(C₁₋₂)alkyl, phenyl(C₁₋₂ alkyl)NH or heteroaryl(C₁₋₂ alkyl)NH; wherein the phenyl and heteroaryl rings of R⁷ are optionally substituted by halo, cyano, nitro, hydroxy, C₁₋₄ alkyl, C₁₋₄ alkoxy, S(O)ₖ(C₁₋₄ alkyl), S(O)₂NR¹²R¹³, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁸ and R¹¹ are, independently, hydrogen, C₁₋₆ alkyl or C₃₋₇ cycloalkyl;
R⁹ is aryl, heteroaryl, C₁₋₆ alkyl, C₃₋₇ cycloalkyl or heterocyclyl;
R¹⁰ aryl, heteroaryl or heterocyclyl;
R¹² and R¹³ are, independently, hydrogen or C₁₋₄ alkyl, or together with a nitrogen or oxygen atom, may join to form a 5- or 6-membered ring which is optionally substituted with C₁₋₄ alkyl, C(O)H, C(O)(C₁₋₄ alkyl) or SO₂(C₁₋₄ alkyl); aryl, phenyl and heteroaryl moieties are independently optionally substituted by one or more of halo, cyano, nitro, hydroxy, OC(O)NR²⁴R²⁵, NR²⁶R²⁷, NR²C(O)R²⁹, NR³⁰C(O)NR³¹R³², S(O)₂NR³³R³⁴, NR³⁵S(O)₂ R³⁶, C(O)NR³⁷R³⁸, CO₂R³⁹, NR⁴⁰CO₂R⁴¹, S(O)_{q}R⁴², OS(O)₂R⁴³, C₁₋₆ alkyl (optionally mono-substituted by S(O)₂R⁴⁴ or C(O)NR⁴⁵R⁴⁶), C₂₋₆ alkenyl, C₂₋₆ alkynyl, C₃₋₁₀ cycloalkyl, C₁₋₆ haloalkyl, C₁₋₆ alkoxy(C₁₋₆)alkyl, C₁₋₆ alkoxy (optionally mono-substituted by CO₂R⁴⁷, C(O)NR⁴⁸R⁴⁹, cyano, heteroaryl or C(O)NHS(O)₂R⁵⁰), NHC(O)NHR⁵¹, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃;
unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁵²R⁵³, C(O)R⁵⁴, C(O)₂(C₁₋₆ alkyl) (such as tert-butoxycarbonyl), C(O)₂(phenyl(C₁₋₂ alkyl)) (such as benzyloxycarbonyl), C(O)NHR⁵⁵, S(O)₂R⁶, NHS(O)₂NHR⁵⁷, NHC(O)R⁵⁸, NHC(O)NHR⁵⁹ or NHS(O)₂R⁶⁰, provided none of these last four substituents is linked to a ring nitrogen;
k, l, p and q are, independently, 0, 1 or 2;
R²⁴, R²⁶, R²⁸, R³⁰, R³¹, R³³, R³⁵, R³⁷, R⁴⁰, R⁵², R⁴⁵ and R⁴⁸ are, independently, hydrogen or C₁₋₆ alkyl;
R²⁵, R²⁷R²⁹R³²R³⁴, R³⁶, R³⁸R³⁹, R⁴¹, R⁴², R⁵³, R⁵⁴, R⁵⁵, R⁵⁶, R⁵⁷R⁵⁸R⁵⁹R⁶⁰, R⁴³, R⁴⁴, R⁴⁶, R⁴⁷, R⁴⁹, R⁵⁰ and R⁵¹ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; R²⁵, R²⁷R²⁹R³², R³⁴, R³⁸, R³⁹, R⁵³R⁵⁴, R⁵⁵, R⁵⁷, R⁵⁸, R⁵⁹, R⁴⁶, R⁴⁷, R⁴⁹ and R⁵¹ may additionally be hydrogen;
or a pharmaceutically acceptable salt thereof;
provided that when R¹ is an optionally substituted isolated 6-membered heterocyclyl and R⁴ is C₁₋₃ alkyl, then R⁵ is not an optionally substituted five membered heterocycle containing at least one carbon atom, one to four nitrogen atoms and, optionally, one oxygen or sulphur atom, said five membered heterocycle being optionally fused to another ring.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, succinate, malonate, tartrate, citrate, oxalate, methanesulphonate or p-toluenesulphonate.

The compounds of the invention may exist as solvates (such as hydrates) and the present invention covers all such solvates.

Alkyl groups and moieties are straight or branched chain and are, for example, methyl, ethyl, n-propyl, iso-propyl, n-butyl, sec-butyl or tert-butyl. Methyl is sometimes abbreviated to Me hereinbelow.

Fluoroalkyl includes, for example, one to six, such as one to three, fluorine atoms, and comprises, for example, a CF₃ group. Fluoroalkyl is, for example, CF₃ or CH₂CF₃.

Cycloalkyl is, for example, cyclopropyl, cyclopentyl or cyclohexyl.

Aryl includes phenyl and naphthyl. In one aspect of the invention aryl is phenyl.

Phenyl(C₁₋₂ alkyl)alkyl is, for example, benzyl, 1-(phenyl)eth-1-yl or 1-(phenyl)eth-2-yl.

Heteroaryl(C₁₋₂ alkyl)alkyl is, for example, pyridinylmethyl, pyrimidinylmethyl or 1-(pyridinyl)eth-2-yl.

Phenyl(C₁₋₂ alkyl)NH is, for example, benzylamino. Heteroaryl(C₁₋₂ alkyl)NH is, for example, pyridinylCH₂NH, pyrimidinylCH₂NH or pyridinylCH(CH₃)NH.

Heteroaryl is an aromatic 5 or 6 membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, pyridinyl, pyrimidinyl, indolyl, benzo[b]furyl (also known as benzfuryl), benz[b]thienyl (also known as benzthienyl or benzthiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl, a naphthyridinyl (for example [1,6]naphthyridinyl or [1,8]naphthyridinyl), a benzothiazinyl or dibenzothiophenyl (also known as dibenzothienyl); or an N-oxide thereof, or an S-oxide or S-dioxide thereof. Heteroaryl can also be pyrazinyl. Heteroaryl is, for example, pyridinyl, pyrimidinyl, indolyl or benzimidazolyl.

Aryloxy includes phenoxy.

Heterocyclyl is, for example, a four, five or six membered ring containing one or two nitrogen, oxygen or sulphur atoms and is, for example, piperidine, piperazine, pyrrolidine, azetidine, tetrahydropyran, tetrahydrothiopyran, tetrahydrothiopyran-S-dioxide, morpholine or thiomorpholine ring.

The five membered heterocycle of R⁵ is, for example, pyrazolyl, imidazolyl, 1,2,4-triazolyl, 1,2,3-triazolyl, tetrazolyl, oxazolyl, isoxazolyl, 1,2,4-oxadiazolyl, 1,3,4-oxadiazolyl or thiazolyl. When the five membered heterocycle of R⁵ is fused to a benzene or pyridine ring the resulting bicyclic is, for example, benzimidazolyl, benztriazolyl or an imidazopyridinyl (such as imidazo[4,5c]pyridinyl). When the five membered ring heterocycle of R⁵ is fused to a saturated cycloalkyl or piperidine the resulting bicyclic is, for example, 4,5,6,7-tetrahydro-1H-benzimidazole, 4,5,6,7-tetrahydro-3H-imidazo[4,5-c]pyridine or 4,5,6,7-tetrahydro-1H-imidazo[4,5-c]pyridine.

In one particular aspect the present invention provides a compound of the invention wherein:

R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, aryl or heteroaryl;

R⁴ is halo, hydroxy, cyano, C₁₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);

R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl, H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring; the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro};

R², R³, A, X, m, n, R⁶, R⁷, R⁹, R10, R¹⁴, R¹⁵, R¹⁶, R¹⁷, R¹⁸, R¹⁹, R²⁰, R²¹, R²² and R²³ are as defined herein; and,

aryl and heteroaryl moieties are independently optionally substituted as recited herein;

or a pharmaceutically acceptable salt thereof.

In another aspect the present invention provides a compound of the invention wherein: R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, heterocyclyl, aryl or heteroaryl;

R⁴ is halo, hydroxy, cyano, C₁₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);

R⁵ is aryl, (CH₂)ₙXR⁹ or (CH₂)ₘ,R¹⁰, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷;

R², R³, A, X, m, n, R⁶, R⁷, R⁹, R10, R¹⁴, R¹⁵, R¹⁶, R¹⁷, R¹⁸, R¹⁹, R²⁰, R²¹, R²² and R²³ are as defined in herein; and,

heterocyclyl, aryl and heteroaryl moieties are independently optionally substituted as recited herein;

or a pharmaceutically acceptable salt thereof.

In yet another aspect the present invention provides a compound of the invention wherein:

R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, heterocyclyl, aryl or heteroaryl;

R⁴ is halo, hydroxy, cyano, C₄₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);

R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl, H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring; the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro};

R², R³, A, X, m, n, R⁶, R⁷, R⁹, R10, R¹⁴, R¹⁵, R¹⁶, R¹⁷, R¹⁸, R¹⁹, R²⁰, R²¹, R²² and R²³ are as defined herein; and,

heterocyclyl, aryl and heteroaryl moieties are independently optionally substituted as recited herein;

or a pharmaceutically acceptable salt thereof.

In another aspect the present invention provides a compound of the invention wherein, unless specified otherwise, aryl, phenyl and heteroaryl moieties are independently optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)[N-linked heterocyclyl], CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, phenyl, heteroaryl, phenyl(C₁₋₄ alkyl), heteroaryl(C₁₋₄ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃.

In another aspect the present invention provides a compound of the invention wherein, unless specified otherwise, aryl, phenyl and heteroaryl moieties are independently optionally substituted by one or more of halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), CF₃, CHF₂, CH₂F, CH₂CF₃ or OCF₃.

In yet another aspect the present invention provides a compound of the invention wherein heterocyclyl is optionally substituted (such as singly substituted for example on a ring nitrogen atom when present) by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)} or heteroaryl {which itself optionally substituted by halo, C₁₋₄ alkyl, C₁₋₄ alkoxy, cyano, nitro, CF₃, (C₄ alkyl)C(O)NH, S(O)₂NH₂, C₁₋₄ alkylthio or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}, S(O)₂NR⁵²R⁵³, C(O)R⁵⁴, C(O)NHR⁵⁵ or S(O)₂R⁵⁶; wherein R⁵², R⁵³, R⁵⁴, R⁵⁵ and R⁵⁶ are, independently, C₁₋₆ alkyl, and R⁵², R⁵³ and R⁵⁵ can also be hydrogen.

In a further aspect of the invention A is absent.

In a still further aspect of the invention R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, aryl or heteroaryl.

In another aspect of the invention R¹⁴, R¹⁷, R¹⁹, R²⁰ and R²² are hydrogen or C₁₋₄ alkyl (for example methyl). In yet another aspect R¹⁴, R¹⁷, R¹⁹, R²⁰ and R²² are hydrogen.

In a further aspect of the invention R¹⁵, R¹⁶, R¹⁸, R²¹, R²² and R²³ are C₁₋₈ alkyl (optionally substituted by halo, C₁₋₆ alkoxy, C₁₋₆ haloalkoxy, C₃₋₆ cycloalkyl (optionally substituted by halo), C₅₋₆ cycloalkenyl, S(O)₂(C₁₋₄ alkyl), heteroaryl, phenyl, heteroaryloxy or aryloxy (for example phenoxy)), phenyl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₄ alkyl), halo or C₁₋₄ alkyl); k is 0, 1 or 2; or R¹⁴ and R¹⁵, and/or R²⁰ and R²¹ may join to form a 4-, 5- or 6-membered ring which optionally includes a nitrogen, oxygen or sulphur atom, said ring being optionally substituted by C₁₋₆ alkyl or C(O)(C₁₋₆ alkyl).

In yet another aspect of the invention R¹⁵, R¹⁶, R¹⁸, R²¹ and R²³ are C₁₋₈ alkyl (optionally substituted by halo (such as fluoro)), phenyl (optionally substituted as recited above), C₃₋₆ cycloalkyl (optionally substituted by halo (such as fluoro)) or C-linked nitrogen containing heterocyclyl (optionally substituted on the ring nitrogen).

In a further aspect R¹ is NR¹⁷C(O)R¹⁸, phenyl or heterocyclyl, wherein R¹⁸ is as defined above, and phenyl and heterocyclyl are optionally substituted as described above. For example R¹⁷ is hydrogen.

In yet another aspect of the invention R¹⁸ is C₁₋₈ alkyl (optionally substituted by halo (such as fluoro, for example to form CF₃CH₂)), phenyl (optionally substituted as recited above), C₃₋₆ cycloalkyl (optionally substituted by halo (such as fluoro, for example to form 1,1-difluorocyclohex-4-yl)) or C-linked nitrogen containing heterocyclyl (such as tetrahydropyran or piperidine, optionally substituted on the ring nitrogen).

In another aspect the present invention provides a compound of the invention wherein R¹⁸ is C₁₋₈ alkyl (optionally substituted by halo (such as fluoro, for example to form CF₃CH₂)), phenyl (optionally substituted by halo) or C₅₋₆ cycloalkyl (optionally substituted by halo (such as fluoro, for example to form 1,1-difluorocyclohex-4-yl)).

In a further aspect of the invention heterocyclyl is optionally substituted (such as singly substituted for example on a ring nitrogen atom when present) by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}, S(O)₂NR⁵²R⁵³, C(O)R⁵⁴, C(O)NHR⁵⁵ or S(O)₂R⁵⁶; wherein R⁵², R⁵³, R⁵⁴, R⁵⁵ and R⁵⁶ are, independently, hydrogen or C₁₋₆ alkyl.

In a still further aspect of the invention R¹ is NR¹⁷C(O)R¹⁸, NR¹⁹C(O)NR²⁰R²¹, NR²²C(O)₂R²³, optionally substituted heterocyclyl, optionally substituted aryl or optionally substituted heteroaryl; wherein R¹⁷, R¹⁸, R¹⁹, R²⁰, R²¹, R²² and R²³ are as defined above; and optional substituents are as defined above.

In yet another aspect of the invention R¹ is optionally substituted aryl (such as optionally substituted phenyl) or optionally substituted heteroaryl, wherein the optional substituents are as recited above.

In a further aspect of the invention when R¹ is optionally substituted heterocyclyl it is, for example, an optionally substituted tetrahydropyran, tetrahydrothiopyran, piperidine, piperazine, pyrrolidine or azetidine. In another aspect when R¹ is optionally substituted heterocyclyl it is, for example, an optionally substituted piperidine, piperazine, pyrrolidine or azetidine (such as an optionally substituted: piperidin-1-yl, piperidin-4-yl, piperazin-1-yl, pyrrolidin-1-yl, pyrrolidin-3-yl, azetidin-1-yl or azetidin-3-yl).

In a still further aspect of the invention the heterocyclyl (for example a ring as described above) of R¹ is mono-substituted by C₁₋₆ alkyl, C₃₋₇ cycloalkyl, phenyl {optionally substituted by halo (for example fluoro), C₁₋₄ alkyl (for example methyl), C₁₋₄ alkoxy (for example methoxy), CF₃ or OCF₃}, S(O)₂(C₁₋₄ alkyl) (for example S(O)₂CH₃, S(O)₂CH₂CH₃ or S(O)₂CH(CH₃)₂), S(O)₂(C₁₋₄ fluoroalkyl) (for example S(O)₂CF₃ or S(O)₂CH₂CF₃), S(O)₂N(C₁₋₄ alkyl)₂, S(O)₂phenyl {optionally substituted (such as mono-substituted) by halo (for example chloro), cyano, C₁₋₄ alkyl, C₁₋₄ alkoxy, CF₃, OCF₃, S(O)₂(C₁₋₄ alkyl) (for example S(O)₂CH₃ or S(O)₂CH₂CH₂CH₃) or S(O)₂(C₁₋₄ fluoroalkyl) (for example S(O)₂CH₂CF₃)}, benzyl {optionally substituted by halo (for example chloro or fluoro), C₁₋₄ alkyl, C₁₋₄ alkoxy (for example methoxy), CF₃ or OCF₃}, C(O)H, C(O)(C₁₋₄ alkyl), benzoyl {optionally substituted by halo (for example chloro or fluoro), C₁₋₄ alkyl (for example methyl), C₁₋₄ alkoxy, CF₃ or OCF₃}, C(O)₂(C₁₋₄ alkyl), C(O)NH₂, C(O)NH(C₁₋₄ alkyl) or C(O)NHphenyl {optionally substituted by halo (for example fluoro), C₁₋₄ alkyl, C₁₋₄ alkoxy, CF₃ or OCF₃}. In a still further aspect when said heterocyclyl is a 4-substituted piperidin-1-yl, a 1-substituted piperidin-4-yl, a 4-substituted piperazin-1-yl, a 3-substituted pyrrolidin-1-yl, a 1-substituted pyrrolidin-3-yl, a 3-substituted azetidin-1-yl or a 1-substituted azetidin-3-yl (for example where said substituent is as recited earlier in this paragraph). In another aspect said heterocyclyl is a 1-substituted piperidin-4-yl or a 4-substituted piperazin-1-yl, wherein the substituent is S(O)₂(C₁₋₄ alkyl), S(O)₂(C₁₋₄ haloalkyl), S(O)₂(phenyl), S(O)₂N(C₁₋₄ alkyl)₂ or phenyl.

In another aspect of the invention R¹ is piperidinyl or piperazinyl (such as piperidin-4-yl or piperazin-1-yl), either of which is N-substituted by phenyl, S(O)₂R⁴² (wherein R⁴² is C₁₋₄ alkyl (such as methyl or ethyl), phenyl or CF₃) or S(O)₂NR³³R³⁴ (wherein R³³ and R³⁴ are, independently, C₁₋₄ alkyl (such as methyl)).

In yet another aspect of the invention R¹ is NHC(O)R¹⁸ is wherein R¹⁸ is C₁₋₄ haloalkyl (for example C₁₋₄ fluoroalkyl, such as CH₂CF₃ or CH₂CH₂CF₃), phenyl (optionally substituted by halo) or C₃₋₆ cycloalkyl (substituted by one or two fluoros).

In a further aspect of the invention R¹ is phenyl optionally substituted by S(O)₂R⁴² (wherein R⁴² is C₁₋₄ alkyl (such as methyl)).

In a still further aspect of the invention R¹ is heteroaryl (such as pyridinyl) optionally substituted by CF₃.

In another aspect of the invention R¹ is heterocyclyl (such as tetrahydropyran, tetrahydrothiopyran or tetrahydrothiopyran-S-dioxide).

In a further aspect the invention provides a compound wherein R¹ is: 1-substituted piperidin-4-yl or a 4-substituted piperazin-1-yl, wherein the substituent is S(O)₂(C₁₋₄ alkyl), S(O)₂(C₁₋₄ haloalkyl), S(O)₂(phenyl), S(O)₂N(C₁₋₄ alkyl)₂ or phenyl; NHC(O)R¹⁸ wherein R¹⁸ is C₁₋₄ haloalkyl, phenyl (optionally substituted by halo) or C₃₋₆ cycloalkyl (substituted by one or two fluoros); phenyl optionally substituted by S(O)₂R⁴² (wherein R⁴² is C₁₋₄ alkyl); or, heterocyclyl (such as tetrahydropyran, tetrahydrothiopyran or tetrahydrothiopyran-S-dioxide).

In another aspect the present invention provides a compound of the invention wherein R² is phenyl or heteroaryl (such as thienyl), either of which is optionally substituted by halo (such as chloro or fluoro), C₁₋₄ alkyl or CF₃.

In yet another aspect of the invention R² is phenyl; phenyl substituted (such as in the 3-, or the 3- and 5-positions) by halo (such as chloro or fluoro) and/or CF₃; or thienyl substituted by halo (such as chloro or fluoro).

In a further aspect of the invention R² is phenyl, 3-fluorophenyl, 3-chlorophenyl, 3-chloro-5-fluorophenyl, 3-trifluoromethylphenyl or 3,5-difluorophenyl. In a still further aspect of the invention R² is phenyl, 3-fluorophenyl or 3,5-difluorophenyl.

In another aspect of the invention R³ is hydrogen or methyl. In a further aspect of the invention when R³ is C₁₋₄ alkyl (such as methyl) the carbon to which R³ is attached has the R absolute configuration. In yet another aspect of the invention R³ is hydrogen.

In a further aspect the invention provides a compound wherein R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) or C(O)N(C₁₋₄ alkyl)₂, then R⁵ can also be NR⁶C(O)R⁷.

In a further aspect of the invention R⁵ is CH₂CH₂S(O)₂R⁹.

In another aspect the present invention provides a compound of the invention wherein R⁹ is optionally substituted aryl (such as phenyl) or optionally substituted heteroaryl (such as pyridyl, imidazolyl or 1,3,4-thiadiazolyl), (the optional substituents being selected from those recited above).

In yet another aspect the present invention provides a compound of the invention wherein R⁹ is phenyl optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, heteroaryl or heteroaryl(C₁₋₄ alkyl); wherein the foregoing heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃ {and in a further aspect of the invention the foregoing heteroaryl groups (such as tetrazolyl) are optionally substituted by C₁₋₄ alkyl}.

In a further aspect the present invention provides a compound of the invention wherein R⁹ is phenyl optionally substituted by halogen (such as chloro or fluoro), cyano, C₁₋₄ alkyl (mono-substituted by S(O)₂(C₁₋₄ alkyl) or C(O)NH(C₁₋₄ alkyl), C₁₋₄ alkoxy, S(C₁₋₄ alkyl), S(O)₂(C₁₋₄ alkyl), OS(O)₂(C₁₋₄ alkyl), OCH₂COOH, OCH₂-tetrazolyl (itself optionally substituted by C₁₋₄ alkyl), carboxamide or tetrazolyl (itself optionally substituted by C₁₋₄ alkyl).

In yet another aspect the present invention provides a compound of the invention wherein R⁹ is aryl or heteroaryl each being optionally substituted by OS(O)₂R⁴³ or C₁₋₆ alkyl (mono-substituted by S(O)₂ R⁴⁴ or C(O)NR⁴⁵R⁴⁶); wherein R⁴³, R⁴⁴, R⁴⁵ and R⁴⁶ are as defined above.

In a further aspect the present invention provides a compound of the invention wherein R⁹ is phenyl (optionally substituted by halogen (such as chloro or fluoro), cyano, C₁₋₄ alkyl, C₁₋₄ alkoxy, S(C₁₋₄ alkyl), S(O)₂(C₁₋₄ alkyl), OS(O)₂(C₁₋₄ alkyl) or carboxamide), C₃₋₇ cycloalkyl (such as cyclohexyl), pyridyl (optionally substituted by C₁₋₄ alkyl), imidazolyl (optionally substituted by C₁₋₄ alkyl) or 1,3,4-thiadiazolyl (optionally substituted by C₁₋₄ alkyl).

In a further aspect the present invention provides a compound of the invention wherein R⁹ is phenyl {optionally substituted by S(O)₂(C₁₋₄ alkyl) (such as CH₃S(O)₂, for example in the 4-position), C₁₋₄ alkoxy (such as CH₃O, for example in the 4-position), OS(O)₂(C₁₋₄ alkyl) (such as OSO₂CH₃, for example in the 4-position), halogen (such as chloro or fluoro) or cyano}.

In another aspect of the invention R⁵ is (CH₂)ₘR¹⁰.

In a further aspect the present invention provides a compound of the invention wherein R¹⁰ is optionally substituted phenyl.

In a still further aspect R¹⁰ is phenyl optionally substituted by halo, C₁₋₄ alkyl, C₁₋₄ alkoxy, S(O)ₛ(C₁₋₄ alkyl), nitro, cyano or CF₃; wherein s is 0, 1 or 2.

In another aspect the present invention provides a compound wherein R⁴ is halo, hydroxy, cyano, C₄₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl).

In yet another aspect of the invention R⁴ is halo (such as fluoro), hydroxy, C₁₋₆ alkyl (such as methyl or ethyl) or C₁₋₆ alkoxy (such as methoxy).

In another aspect of the invention R⁴ is halo (such as fluoro), hydroxy, C₄₋₆ alkyl or C₁₋₆ alkoxy (such as methoxy).

In yet another aspect of the invention R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) or C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl, H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring; the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro}.

In a further aspect of the invention R⁵ is NR⁶C(O)R⁷.

In a still further aspect the present invention provides a compound of the invention wherein R⁶ is ethyl.

In another aspect of the invention R⁷ is phenyl(C₁₋₂)alkyl, phenyl(C₁₋₂ alkyl)NH, phenyl, heteroaryl or heteroaryl(C₁₋₂)alkyl; wherein the phenyl and heteroaryl rings are optionally substituted by halo, cyano, nitro, hydroxy, C₁₋₄ alkyl, C₁₋₄ alkoxy, S(O)ₖC₁₋₄ alkyl, S(O)₂NR¹²R¹³, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; and R¹² and R¹³ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl); and k is 0, 1 or 2 (for example, 2).

In another aspect the invention provides a compound of the invention wherein R⁷ is phenyl(C₁₋₂)alkyl or phenyl(C₁₋₂ alkyl)NH; wherein the phenyl rings of R⁷ are optionally substituted by halo, cyano, nitro, hydroxy, C₁₋₄ alkyl, C₁₋₄ alkoxy, S(O)ₖC₁₋₄ alkyl, S(O)₂NR¹²R¹³, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; R¹² and R¹³ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl); and k is 0, 1 or 2.

In another aspect R⁷ is phenyl or benzyl; wherein the aromatic rings are optionally substituted by halo, cyano, nitro, hydroxy, C₁₋₄ alkyl, C₁₋₄ alkoxy, S(O)ₖC₁₋₄ alkyl, S(O)₂NR¹²R¹³, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; k is 0, 1 or 2; and R¹² and R¹³ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl).

In a further aspect R⁷ is phenyl, benzyl or NHCH₂phenyl (such as benzyl); wherein the phenyl rings are optionally substituted by halo, cyano, nitro, hydroxy, C₁₋₄ alkyl, C₁₋₄ alkoxy, S(O)₂C₁₋₄ alkyl, S(O)₂NR¹²R¹³, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃; and R¹² and R¹³ are, independently, hydrogen or C₁₋₄ alkyl.

In yet another aspect R⁷ is benzyl or NHCH₂phenyl (such as benzyl) wherein the phenyl rings are optionally substituted by halo (such as fluoro, chloro or bromo), cyano, C₁₋₄ alkyl (such as methyl), C₁₋₄ alkoxy (such as methoxy) or S(O)₂C₁₋₄ alkyl (such as S(O)₂CH₃).

In a still further aspect R⁷ is phenyl, benzyl or NHCH₂phenyl, wherein the phenyl rings are substituted (for example in the para-position) by S(O)₂C₁₋₄ alkyl and the rings are optionally further substituted by halo, cyano, nitro, hydroxy, C₁₋₄ alkyl or C₁₋₄ alkoxy.

In another aspect R⁷ is benzyl, wherein the phenyl ring is substituted (for example in the para-position) by S(O)₂C₁₋₄ alkyl (such as S(O)₂CH₃); R⁷ is, for example, CH₂(4-S(O)₂CH₃—C₆H₄).

In yet another aspect R⁵ is 1,2,4-triazolyl, thiazolyl, 1,2,4-oxadiazolyl, imidazolyl or 1,2,3-triazolyl substituted as described above. In a further aspect R⁵ is 1,2,4-triazolyl, thiazolyl, 1,2,4-oxadiazolyl, benzimidazolyl, benztriazolyl or an imidazopyridinyl (such as imidazo[4,5c]pyridinyl), each of which is unsubstituted or substituted by one or two of the same or different C₁₋₆ alkyl (for example C₁₋₄ alkyl; such as methyl), CF₃, OH (which may tautomerise to the keto form), S(O)₂(C₁₋₄ alkyl), C(O)NH₂, C(O)NH(phenyl(C₁₋₂ alkyl)) or phenyl(C₁₋₂ alkyl); wherein the phenyl of the foregoing phenyl(C₁₋₂ alkyl) groups is optionally substituted by halo, C₁₋₄ alkyl, C₁₋₄ alkoxy, cyano or S(O)₂(C₁₋₄ alkyl).

In a further aspect the present invention provides a compound of formula (I) wherein A is absent; R¹ is phenyl [optionally substituted by S(O)₂(C₁₋₄ alkyl) (for example S(O)₂CH₃)], NHC(O)(4,4-difluorocyclohexyl), piperidin-4-yl [N-substituted by S(O)₂(C₁₋₄ alkyl) (for example S(O)₂CH₃)], tetrahydropyranyl or tetrahydrothiopyranyl-S-dioxide; R² is phenyl or phenyl optionally substituted by halo (for example fluoro); R³ is hydrogen; R⁴ is halo (such as fluoro), hydroxy, C₁₋₆ alkyl (such as methyl or ethyl) or C₁₋₆ alkoxy (such as methoxy); R⁵ is phenyl (optionally substituted by halo (such as chloro)), CH₂CH₂S(O)₂R⁹ or NHC(O)R⁷; R⁷ is CH₂phenyl optionally substituted by S(O)₂(C₁₋₄ alkyl) (for example S(O)₂CH₃); and, R⁹ is phenyl optionally substituted by S(O)₂(C₁₋₄ alkyl) (for example S(O)₂CH₃).

In a still further aspect the present invention provides a compound of formula (Ia): 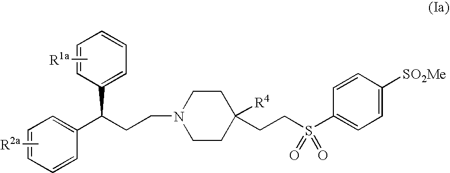
wherein R⁴ is as defined above; R^{1a} is one or more of the same or different phenyl substituents as defined above; and, R^{2a} is one or two halogen atoms (such as fluoro), or a CF₃ group.

In another aspect the present invention provides a compound of formula (Ib): 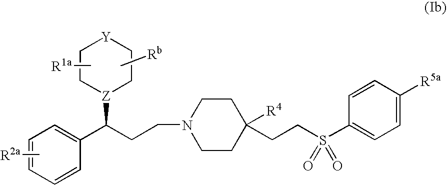
wherein R^{2a} and R⁴ are as defined above; R^{a} and R^{b} are, independently, hydrogen or C₁₋₄ alkyl; Y is oxygen, sulphur, sulphur dioxide or N(S(O)₂(C₁₋₄ alkyl)); Z is CH, N or C(C₁₋₄ alkyl) (for example Z is CH); and R^{5a} is S(O)₂(C₁₋₄ alkyl) or C₁₋₄ alkoxy (for example R^{5a}is S(O)₂CH₃).

In yet another aspect the present invention provides a compound of formula (Ic): 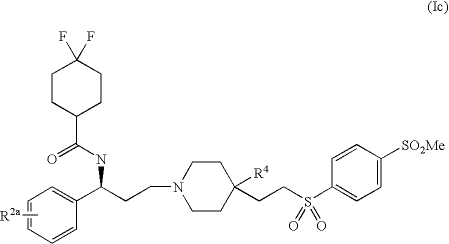
wherein R^{2a} and R⁴ are as defined above.

In a further aspect the present invention provides a compound of formula (Id): 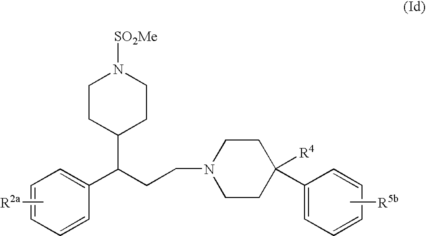
wherein R^{2a} and R⁴ are as defined above; and R^{5b}is one or more of the same or different phenyl substituents as defined above.

In a still further aspect the present invention provides a compound of formula (Ie): 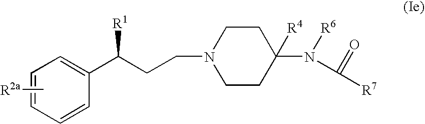
wherein R¹, R^{2a}, R⁴, R⁶ and R⁷ are as defined above.

In another aspect the present invention provides a compound of formula (If): 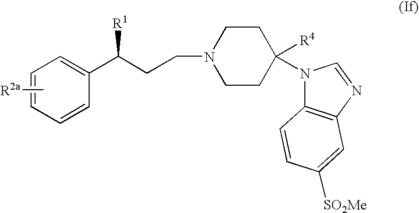
wherein R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, aryl or heteroaryl; and R^{2a} and R⁴ are as defined above.

In yet another aspect the present invention provides a compound of formula (Ig): 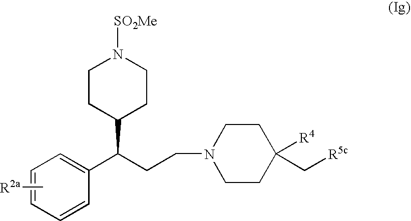
wherein R^{2a} and R⁴ are as defined above, and R^{5c} is optionally substituted phenyl (the optional substituents being as defined above, for example S(O)₂(C₁₋₄ alkyl)) or optionally substituted heteroaryl (the optional substituents being as defined above, for example C₁₋₄ alkyl).

In a further aspect the present invention provides a compound of formula (Ih): 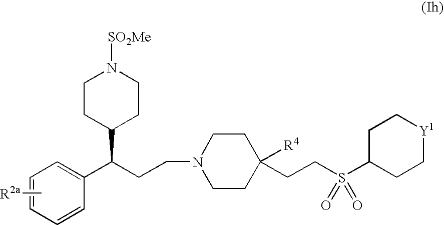
wherein R^{2a} and R⁴ are as defined above, and Y¹ is O, S, S(O)₂, NS(O)₂NR⁵²R⁵³, NC(O)R⁵⁴, NC(O)₂(C₁₋₆ alkyl), NC(O)₂(phenyl(C₁₋₂ alkyl)), NC(O)NHR⁵⁵ or NS(O)₂R⁵⁶; wherein R⁵², R⁵³, R⁵⁴, R⁵⁵ and R⁵⁶ are as defined above (for example they are, independently, C₁₋₆ alkyl, and R⁵², R⁵³ and R⁵⁵ can also be hydrogen).

In a still further aspect the present invention provides a compound of formula (Ih): 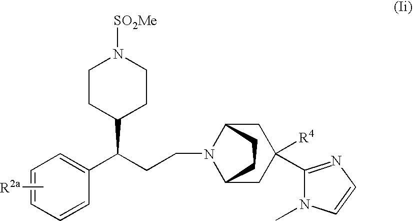
wherein R^{2a} is as defined above, and R⁴ is halo, hydroxy; cyano, C₄₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl).

The compounds in the following Tables illustrate the invention.

TABLE I
Table I comprises compounds of formula (Ia):

| | |
|---|---|
| | (Ia) |
| 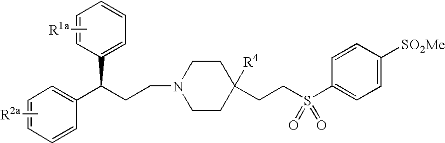 | |

| Compound No. | R^{1a} | R^{2a} | R⁴ | LCMS (MH⁺) |
|---|---|---|---|---|
| 1 | 4-SO₂Me | 3,5-F₂ | CH₃ | 654 |
| 2 | 4-SO₂Me | 3,5-F₂ | OH | 656 |
| 3 | 4-SO₂Me | 3,5-F₂ | OMe | 670 |
| 4 | 4-SO₂Me | 3,5-F₂ | F | 658 |
| 5 | 4-SO₂Me | 3,5-F₂ | Et | 668 |
| 6 | 4-SO₂Me | 3,5-F₂ | CN | 665 |

TABLE II
Table II comprises compounds of formula (Ib):

| | |
|---|---|
| | (Ib) |
| 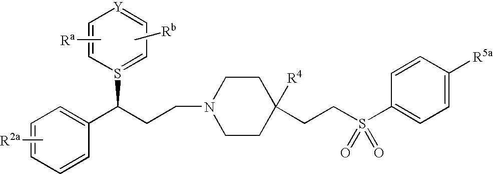 | |

| Compound | | | | | | | | LCMS |
|---|---|---|---|---|---|---|---|---|
| No | Y | Z | R^{2a} | R⁴ | R^{5a} | R^{a} | R^{b} | (MH⁺) |
| 1 | NSO₂Me | CH | H | CH₃ | SO₂Me | H | H | 625 |
| 2 | O | CH | 3,5-F₂ | F | SO₂Me | H | H | 588 |
| 3 | NSO₂Me | CH | 3,5-F₂ | F | SO₂Me | H | H | 665 |
| 4 | SO₂ | CH | 3,5-F₂ | F | SO₂Me | H | H | 636 |
| 5 | O | CH | 3,5-F₂ | CH₃ | SO₂Me | H | H | 584 |
| 6 | NSO₂Me | CH | 3,5-F₂ | CH₃ | SO₂Me | H | H | 660 |
| 7 | NSO₂Me | CH | 3,5-F₂ | OH | SO₂Me | H | H | 663 |
| 8 | O | CH | 3,5-F₂ | OH | SO₂Me | H | H | 586 |
| 9 | O | CH | 3,5-F₂ | CH₃ | SO₂Me | 2-CH₃(S) | H | 598 |
| 10 | O | CH | 3,5-F₂ | F | SO₂Me | 2-CH₃(S) | H | 602 |
| 11 | SO₂ | CH | 3,5-F₂ | CH₃ | SO₂Me | H | H | 632 |
| 12 | NSO₂Me | CH | 3,5-F₂ | Et | SO₂Me | H | H | 675 |
| 13 | NSO₂Me | CH | 3,5-F₂ | OMe | SO₂Me | H | H | 677 |
| 14 | NSO₂Me | CH | 3,5-F₂ | CN | SO₂Me | H | H | 672 |
| 15 | O | CH | 3,5-F₂ | OMe | SO₂Me | H | H | 600 |
| 16 | NSO₂Me | CH | 3,5-F₂ | OMe | OMe | H | H | 629 |
| 17 | NSO₂Me | CH | 3,5-F₂ | OH | OMe | H | H | 615 |
| 18 | NSO₂Me | N | H | CH₃ | SO₂Me | H | H | 626 |
| 19 | NSO₂Me | N | 3,5-F₂ | CH₃ | SO₂Me | H | H | 662 |
| 20 | NSO₂Me | N | 3,5-F₂ | CH₃ | OMe | H | H | 614 |
| 21 | O | C(CH₃) | 3,5-F₂ | CN | SO₂Me | H | H | 609 |

TABLE III
Table III comprises compounds of formula (Ic):

| | |
|---|---|
| | (Ic) |
| 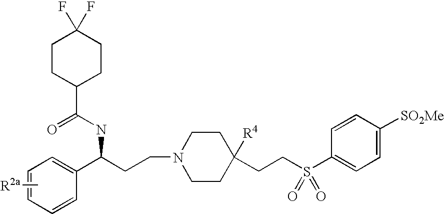 | |

| Compound No | R^{2a} | R⁴ | LCMS (MH⁺) |
|---|---|---|---|
| 1 | H | F | 629 |
| 2 | H | OH | 627 |
| 3 | H | CH₃ | 625 |

TABLE IV
Table IV comprises compounds of formula (Id):

| | |
|---|---|
| | (Id) |
| 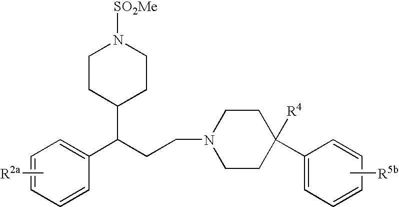 | |

| Compound No | R^{2a} | R⁴ | R^{5b} | LCMS (MH⁺) |
|---|---|---|---|---|
| 1 | H | OH | H | 457 |
| 2 | H | OH | 4-Cl | 491/493 |

TABLE V
Table V comprises a compound of formula (Ie):

| | |
|---|---|
| | (1e) |
| 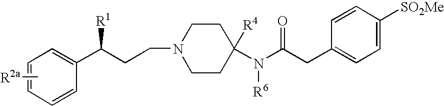 | |

| | | | | | LCMS |
|---|---|---|---|---|---|
| Compound No | R¹ | R^{2a} | R⁴ | R⁶ | (MH⁺) |
| 1 | 4-SO₂MePh | 3,5-F₂ | Et | H | 633 |
| 2 | 4-(Piperidine-1-S(O)₂Me) | 3,5-F₂ | Me | Et | 654 |

TABLE VI
Table VI comprises compounds of formula (If):

| | |
|---|---|
| | (If) |
| 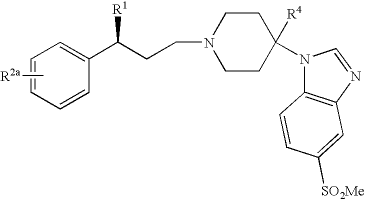 | |

| Compound No | R¹ | R^{2a} | R⁴ | LCMS (MH⁺) |
|---|---|---|---|---|
| 1 | 4-SO₂MePh | 3,5-F₂ | CH₃ | 602 |

TABLE VII
Table VII comprises compounds of formula (Ig):

| | |
|---|---|
| | (Ig) |
| 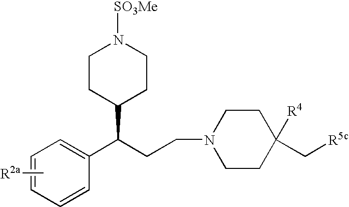 | |

| Compound No | R^{2a} | R⁴ | R^{5c} | LCMS (MH⁺) |
|---|---|---|---|---|
| 1 | 3,5-F₂ | OH | 2-(1-Me-imidazole) | 511 |
| 2 | 3,5-F₂ | OH | 4-SO₂MePh | 584 |

TABLE VIII
Table VIII comprises compounds of formula (Ih):

| | |
|---|---|
| | (Ih) |
| 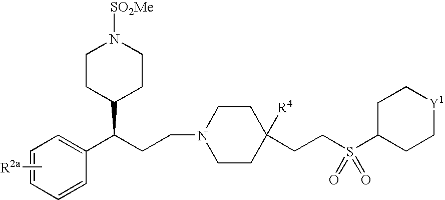 | |

| Compound No | R^{2a} | R⁴ | Y¹ | LCMS (MH⁺) |
|---|---|---|---|---|
| 1 | 3,5-F₂ | CH₃ | NCO₂CH₂Ph | 724 |
| 2 | 3,5-F₂ | CH₃ | NSO₂Me | 668 |
| 3 | 3,5-F₂ | CH₃ | O | 591 |

TABLE IX
Table IX comprises compounds of formula (Ii):

| | |
|---|---|
| | (Ii) |
| 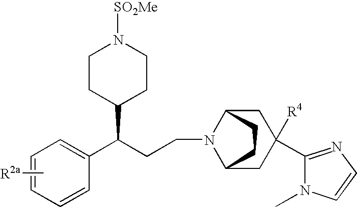 | |

| Compound No | R^{2a} | R⁴ | LCMS (MH⁺) |
|---|---|---|---|
| 1 | 3,5-F₂ | OH | 523 |

In yet another aspect the invention provides each individual compound listed in the Tables above; or a pharmaceutically acceptable salt thereof.

The compounds of formula (I), (Ia), (Ib), (Ic), (Id), (Ie), (If), (Ig), (Ih) and (Ii) can be prepared by methods described below; by routine adaptation of the Examples; or by methods described, or by routine adaptation of methods described, in the patent or other scientific literature.

A compound of the invention can be prepared by reductive amination of a compound of formula (II): 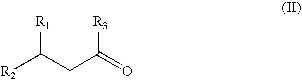
wherein R¹, R² and R³ are as defined above, with a compound of formula (III): 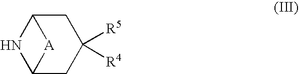
wherein R⁴, R⁵ and A are as defined above, in the presence of NaBH(OAc)₃ in a suitable solvent (such as a chlorinated solvent, for example dichloromethane) and, for example, at room temperature (for example 10-30° C.). Compounds of formula (II) can be prepared by methods described, or by routine adaptation of methods described, in the patent or other scientific literature (for example WO 01/66525, WO 01/87839, WO 02/070479, WO 03/042177, WO 03/042205, WO 03/042178 and EP-A-1013276).

A compound of the invention can also be prepared by the alkylation of a compound of formula (III) with a compound of formula (V): 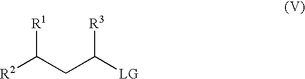
wherein R¹, R² and R³ are as defined above and LG is a leaving group such as, but not restricted to, halide, mesylate, tosylate or triflate, in the presence of a suitable base, such as potassium carbonate or a tertiary amine (for example Hünigs base or triethylamine), in a suitable solvent, such as acetonitrile or THF at a suitable temperature (such as room temperature (for example 10-30° C.)). Compounds of formula (V) can be prepared by methods described, or by routine adaptation of methods described, in the patent or other scientific literature.

A compound of formula (III) can be prepared by removal of the protecting group (PG) from a compound of formula (IV): 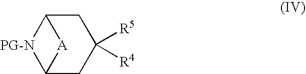
wherein PG is, for example, benzyloxycarbonyl or benzyl tert-butyloxycarbonyl. When PG is benzyloxycarbonyl or benzyl removal can be effected by hydrogenation (for example hydrogen in the presence of palladium on carbon catalyst); when PG is tert-butyloxycarbonyl removal may be effected by treatment with acid (such as hydrochloric acid or trifluoroacetic acid).

In the processes described suitable protecting groups and details of processes for adding and removing such groups may be found in “Protective Groups in Organic Synthesis”, 3rd Edition (1999) by Greene and Wuts.

A compound of formula (IV) can be prepared by methods described, or by routine adaptation of methods described, in the patent or other scientific literature; or, alternatively, certain compounds of formula (IV) can be prepared by a process as described in Scheme 1, 2 or 3. The product of Scheme 4 can be used to prepare compounds of formula (IV) using methods known in the art. Throughout the Schemes: PG is a protecting group and LG is a leaving group both, for example, as defined above; Boc is tert-butoxycarbonyl; mCPBA is meta-chloroperoxybenzoic acid; R* is alkyl; and, DAST is diethylaminosulphur trifluoride.

In a further aspect the present invention provides processes for preparing a compound of formula (I), (Ia), (Ib), (Ic), (Id), (Ie), (If), (Ig), (Ih) or (Ii).

A compound of the invention, or a pharmaceutically acceptable salt thereof, can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,

15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema; in a warm blooded animal, such as man.

The compounds of the invention have activity as pharmaceuticals, in particular as modulators (such as agonists, partial agonists, inverse agonists or antagonists) of chemokine receptor (for example CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia), (Ib), (Ic), (Id), (Ie), (If), (Ig), (Ih) or (Ii), or a pharmaceutically acceptable salt thereof, for use in a method of treatment of a warm blooded animal (such as man) by therapy (including prophylaxis).

According to a further feature of the present invention there is provided a method for modulating chemokine receptor activity (for example CCR5 receptor activity) in a warm blooded animal, such as man, in need of such treatment, which comprises administering to said animal an effective amount of a compound of the present invention, or a pharmaceutically acceptable salt thereof.

The present invention also provides the use of a compound of the formula (I), (Ia), (Ib), (Ic), (Id), (Ie), (If), (Ig), (Ih) or (Ii), or a pharmaceutically acceptable salt thereof, as a medicament, for example as a medicament for the treatment of transplant rejection, respiratory disease, psoriasis or rheumatoid arthritis (such as rheumatoid arthritis). [Respiratory disease is, for example, COPD, asthma {such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis}; and is particularly asthma or rhinitis].

In another aspect the present invention provides the use of a compound of the formula (I), (Ia), (Ib), (Ic), (Id), (Ie), (If), (Ig), (Ih) or (Ii), or a pharmaceutically acceptable salt thereof, in the manufacture of a medicament for use in therapy (for example modulating chemokine receptor activity (for example CCR5 receptor activity (such as rheumatoid arthritis)) in a warm blooded animal, such as man).

The invention also provides a compound of the formula (I), (Ia), (Ib), (Ic), (Id), (Ie), (If), (Ig), (Ih) or (Ii), or a pharmaceutically acceptable salt thereof, for use as a medicament, for example as a medicament for the treatment of rheumatoid arthritis.

In another aspect the present invention provides the use of a compound of the formula (I), (Ia), (Ib), (Ic), (Id), (Ie), (If), (Ig), (Ih) or (Ii) or a pharmaceutically acceptable salt thereof, in the manufacture of a medicament for use in therapy (for example modulating chemokine receptor activity (for example CCR5 receptor activity (such as rheumatoid arthritis)) in a warm blooded animal, such as man).

The invention further provides the use of a compound of formula (I), (Ia), (Ib), (Ic), (Id), (Ie), (If), (Ig), (1 h) or (Ii), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,

15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema; in a warm blooded animal, such as man.

In another aspect the invention further provides the use of a compound of formula (I), (Ia), (Ib), (Ic), (Id), (Ie), (If), (Ig), (Ih) or (Ii), or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle; in a warm blooded animal, such as man.

The present invention further provides a method of treating a chemokine mediated disease state (for example a CCR5 mediated disease state) in a warm blooded animal, such as man, which comprises administering to a mammal in need of such treatment an effective amount of a compound of formula (I), (Ia), (Ib), (Ic), (Id), (Ie), (If), (Ig), (Ih) or (Ii), or a pharmaceutically acceptable salt thereof.

In order to use a compound of the invention, or a pharmaceutically acceptable salt thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia), (Ib), (Ic), (Id), (Ie), (If), (Ig), (Ih) or (Ii), or a pharmaceutically acceptable salt thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will comprise from 0.05 to 99% w (percent by weight), such as from 0.05 to 80% w, for example from 0.10 to 70% w (such as from 0.10 to 50% w) of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), (Ia), (Ib), (Ic), (Id), (Ie), (If), (Ig), (Ih) or (Ii), or a pharmaceutically acceptable salt thereof (hereafter Compound X), for therapeutic or prophylactic use in humans:

(a)
Tablet I

| | mg/tablet |
|---|---|
| Compound X | 100 |
| Lactose Ph. Eur. | 179 |
| Croscarmellose sodium | 12.0 |
| Polyvinylpyrrolidone | 6 |
| Magnesium stearate | 3.0 |

(b)
Tablet II

| | mg/tablet |
|---|---|
| Compound X | 50 |
| Lactose Ph. Eur. | 229 |
| Croscarmellose sodium | 12.0 |
| Polyvinylpyrrolidone | 6 |
| Magnesium stearate | 3.0 |

(c)
Tablet III

| | mg/tablet |
|---|---|
| Compound X | 1.0 |
| Lactose Ph. Eur. | 92 |
| Croscarmellose sodium | 4.0 |
| Polyvinylpyrrolidone | 2.0 |
| Magnesium stearate | 1.0 |

(d)
Capsule

| | mg/capsule |
|---|---|
| Compound X | 10 |
| Lactose Ph. Eur. | 389 |
| Croscarmellose sodium | 100 |
| Magnesium stearate | 1.0 |

(e)
Injection I

| | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGF,); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; or (xxvii) inhibitor of transcription factor activation such as NFkB, API, or STATS.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies; or

(x) a compound useful in the treatment of AIDS and/or HIV infection for example: an agent which prevents or inhibits the viral protein gp120 from engaging host cell CD4 {such as soluble CD4 (recombinant); an anti-CD4 antibody (or modified/recombinant antibody) for example PRO542; an anti-group120 antibody (or modified/recombinant antibody); or another agent which interferes with the binding of group120 to CD4 for example BMS806}; an agent which prevents binding to a chemokine receptor, other than CCR5, used by the HIV virus {such as a CXCR4 agonist or antagonist or an anti-CXCR4 antibody}; a compound which interferes in the fusion between the HIV viral envelope and a cell membrane {such as an anti-group 41 antibody; enfuvirtide (T-20) or T-1249}; an inhibitor of DC-SIGN (also known as CD209) {such as an anti-DC-SIGN antibody or an inhibitor of DC-SIGN binding}; a nucleoside/nucleotide analogue reverse transciptase inhibitor {for example zidovudine (AZT), nevirapine, didanosine (ddI), zalcitabine (ddC), stavudine (d4T), lamivudine (3TC), abacavir, adefovir or tenofovir (for example as free base or as disoproxil fumarate)}; a non-nucleoside reverse transciptase inhibitor {for example nevirapine, delavirdine or efavirenz}; a protease inhibitor {for example ritonavir, indinavir, saquinavir (for example as free base or as mesylate salt), nelfinavir (for example as free base or as mesylate salt), amprenavir, lopinavir or atazanavir (for example as free base or as sulphate salt)}; a ribonucleotide reductase inhibitor {for example hydroxyurea}; or an antiretroviral {for example emtricitabine}.

The invention will now be illustrated by the following non-limiting Examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10g or 20g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 400 MHz using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺and

(xi) the following abbreviations are used:

DMSO dimethyl sulfoxide;
DMF N,N-dimethylformamide;
DCM dichloromethane;
THF tetrahydrofuran;
DIPEA N,N-diisopropylethylamine;
HATU O-(7-Azabenzotriazol-1-yl)-N,N,N′,N′-tetramethyluronium hexafluorophosphate;
TMEDA N,N,N′N′-tetramethylethylenediamine;
EDTA ethylaminediaminetetraacetic acid; and,
DPPA diphenylphosphoryl azide

### EXAMPLE 1

This Example illustrates the preparation of 1-{(3R)-3-(3,5-difluorophenyl)-3-[4-(methylsulfonyl)phenyl]propyl}-4-methyl-4-(2-{[4-(methylsulfonyl)phenyl]sulfonyl}ethyl)piperidine (Compound No. 1, Table I).

To a solution of (3R)-3-(3,5-difluorophenyl)-3-[4-(methylsulfonyl)phenyl]propanal (170 mg; Method B) in dichloromethane (10 ml) was added 4-methyl-4-(2-{[4-(methylsulfonyl)phenyl]sulfonyl}ethyl)piperidine (198 mg; Method H) and triethylamine (73 μL) followed by MP-Triacetoxyborohydride resin (628 mg, 2.07 mmol/g). The resulting mixture was stirred at room temperature for 18 hours. The mixture was filtered and the organics were washed with saturated sodium bicarbonate, dried (MgSO₄) and evaporated to dryness. The residue was purified on a 20g silica cartridge eluting with a 0 to 5% methanol in ethyl acetate gradient to give the title compound as a white foam (192 mg).

NMR (CDCl₃): 0.95 (s, 3H), 1.35 (m, 4H), 2.7 (m, 2H), 2.2 (m, 6H), 2.4 9m, 2H), 3.05 (s, 3H), 3.1 (m, 2H), 3.15 (s, 3H), 4.1 9m, 1H), 6.6-6.8 (m, 3H), 7.4 (d, 2H), 7.9 (d, 2H), 8.15 (dd, 4H).

The procedure described in Example 1 can be repeated using different aldehydes {such as (3S)-3-[4-(methylsulfonyl)phenyl]-3-phenylpropanal (Method A), (3R)-3-(3,5-difluorophenyl)-3-[1-(methylsulfonyl)piperidin-4-yl]propanal (Method C), (3R)-3-(3,5-difluorophenyl)-3-(tetrahydro-2H-pyran-4-yl)propanal (Method D), (3R)-3-(3,5-difluorophenyl)-3-[(2S)-2-methyltetrahydro-2H-pyran-4-yl]propanal (Method E),3-phenyl-3-(N-methanesulphonylpiperidin-4-yl)propionaldehyde (Method F), 4,4-difluoro-N-[(1s)-3-oxo-1-phenylpropyl]cyclohexanecarboxamide (Method G), (3R)-3-(3,5-difluorophenyl)-3-(1,1-dioxidotetrahydro-2H-thiopyran-4-yl)propanal (Method O) or 4-methyl-tetrahydro-pyran-4-carboxaldehyde (Method S)} in place of (3R)-3-(3,5-difluorophenyl)-3-[4-(methylsulfonyl)phenyl]propanal; or different piperidines or piperidine hydrochlorides {such as 4-methyl-4-(2-{[4-(methylsulfonyl)phenyl]sulfonyl}ethyl)piperidine (Method H), 4-(2-{[4-(methylsulfonyl)phenyl]sulfonyl}ethyl)piperidin-4-ol (Method I), 4-fluoro-4-(2-{[4-(methylsulfonyl)phenyl]sulfonyl}ethyl)piperidine (Method J), 4-methoxy-4-(2-{[4-(methylsulfonyl)phenyl]sulfonyl}ethyl)piperidine (Method K), N-(4-ethylpiperidin-4-yl)-2-[4-(methylsulfonyl)phenyl]acetamide (Method L), 4-phenylpiperin-4-ol (CAS40807-61-2), 4-(4-chlorophenyl)piperidin-4-ol (CAS 39512-49-7), 4-[(1-methyl-1H-imidazol-2-yl)methyl]piperidin-4-ol (Method M), 4-[4-(methylsulfonyl)benzyl]piperidin-4-ol (Method N), (3-endo)-3-(1-methyl-1H-imidazol-2-yl)-8-azabicyclo[3.2.1]octan-3-ol (Method P), N-ethyl-N-(4-methylpiperidin-4-yl)-2-[4-(methylsulfonyl)piperidin-1-yl]acetamide (Method Q), 1-(4-methylpiperidin-4-yl)-5-(methylsulfonyl)-1H-benzimidazole (Method R) benzyl 4-{[2-(4-methylpiperidin-4-yl)ethyl]sulfonyl} piperidine-1-carboxylate (Mehtod T) or 4-(2-{[4-(methylsulfonyl)phenyl]sulfonyl}ethyl)piperidine-4-carbonitrile (Method U)} in place of 4-methyl-4-(2-{[4-(methylsulfonyl)phenyl]sulfonyl}ethyl)piperidine.

### EXAMPLE 2

This Example illustrates the preparation of 1-{(1S)-3-[4-methyl-4-(2-{[4-(methylsulfonyl)phenyl]sulfonyl}ethyl)piperidin-1-yl]-11-phenylpropyl)-4-(methylsulfonyl)piperazine (Compound No. 18, Table II)

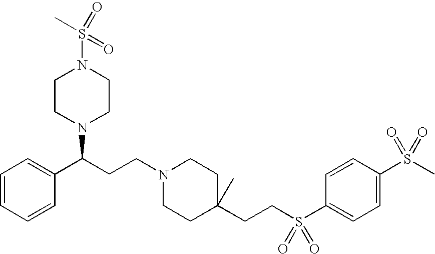

### Step 1: Preparation of (1S)-3-[4-methyl-4-(2-{[4-(methylsulfonyl)phenyl]sulfonyl}ethyl)piperidin-1-yl]-1-phenylpropan-1-ol

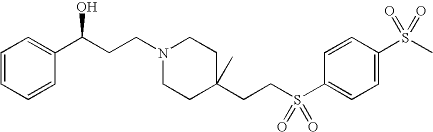

To a mixture of 4-methyl-4-(2-{[4-(methylsulfonyl)phenyl]sulfonyl}ethyl)piperidine (Method H; 382 mg, 1 mmol) and (3S)-3-hydroxy-3-phenylpropyl 4-methylbenzenesulfonate (306 mg, 1 mmol) in dioxane was added potassium carbonate (415 mg, 3 mmol) and the resulting mixture was heated to reflux for 5 hours under a blanket of argon. The reaction was allowed to cool and then concentrated in vacuo. The residue was partitioned between DCM/water (50 ml/50 ml) and the organic layer separated, washed with water (50 ml), brine (50 ml), dried over magnesium sulphate, filtered and then concentrated in vacuo. The resulting foam was purified by flash chromatography using a gradient elution of 0 to 30% methanol in ethyl acetate to give a white solid (296 mg).

NMR (CDCl₃): 0.91 (s, 3H), 1.44 (m, 4H), 1.69 (m, 2H), 1.84 (m, 2H), 2.29 (m, 1H), 2.45 (m, 1H), 2.57 (m, 2H), 2.70 (m, 2H), 3.09 (m, 2H), 3.12 (s, 3H), 4.90 (m, 1H), 7.23 (m, 1H), 7.32 (m, 4H), 8.13 (d, 2H), 8.18 (d, 2H); M+H 480.

### Step 2: Preparation of Title Compound

To a solution of (1S)-3-[4-methyl-4-(2-{[4-(methylsulfonyl)phenyl]sulfonyl}ethyl)-piperidin-1-yl]-1-phenylpropan-1-ol (278 mg, 0.58 mmol) in DCM (6 ml) at 0° C. under a blanket of argon was added triethylamine (161 μl, 1.16 mmol) and methanesulfonyl chloride (69 μl, 0.87 mmol). The mixture was allowed to warm to ambient temperature and stirred overnight, diluted with DCM (25 ml) and then washed with saturated ammonium chloride solution (2×25 ml), brine (25 ml), dried over magnesium sulphate, filtered and concentrated in vacuo. The residue was dissolved in DCM (6 ml) and triethylamine (161 μl, 1.16 mmol) and methanesulfonyl piperazine (190 mg, 1.16 mmol) added and the reaction stirred for 5 days, diluted with DCM (25 ml) and then washed with saturated ammonium chloride solution (2×25 ml), brine (25 ml), dried over magnesium sulphate, filtered and concentrated in vacuo. The crude product was purified by flash chromatography using a gradient elution of 10 to 15% methanol in ethyl acetate to give a white foam (147 mg).

NMR (CDCl₃): 0.84 (s, 3H), 1.39 (m, 4H), 1.66 (m, 2H), 1.89 (m, 1H), 2.07-2.56 (m, 12H), 2.73 (s, 3H), 3.07 (m, 2H), 3.11 (s, 3H), 3.17 (m, 3H), 3.40 (m, 1H), 7.18 (d, 2H), 7.29 (m, 3H), 8.11 (d, 2H), 8.16 (d, 2H).

In a similar manner but using (1S)-3-chloro-1-(3,5-difluorophenyl)propan-1-ol (Method V) in Step 1 was prepared 1-{(1S)-1-(3,5-difluorophenyl)-3-[4-methyl-4-(2-{[4-(methylsulfonyl)phenyl]sulfonyl}ethyl)piperidin-1-yl]propyl}-4-(methylsulfonyl)piperazine (Compound No. 19, Table II).

In a similar manner but using (1S)-3-chloro-1-(3,5-difluorophenyl)propan-1-ol (Method V) and 4-{2-[(4-methoxyphenyl)sulfonyl]ethyl}-4-methylpiperidine in Step 1 was prepared 1-[(1S)-1-(3,5-difluorophenyl)-3-(4-{2-[(4-methoxyphenyl)sulfonyl]ethyl}-4-methylpiperidin-1-yl)propyl]-4-(methylsulfonyl)piperazine (Compound No. 20, Table II).

### EXAMPLE 3

This Example illustrates the preparation 1-{(3R)-3-(3,5-difluorophenyl)-3-[1-(methylsulfonyl)piperidin-4-yl]propyl}-4-methyl-4-(2-{[1-(methylsulfonyl)piperidin-4-yl]sulfonyl}ethyl)piperidine (Compound No. 2, Table VIII).

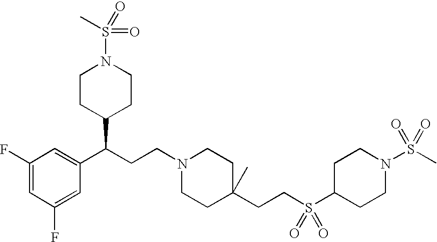

### Step 1: Preparation of 1-{(3R)-3-(3,5-difluorophenyl)-3-[1-(methylsulfonyl)piperidin-4-yl]propyl}-4-methyl-4-[2-(piperidin-4-ylsulfonyl)ethyl]piperidine

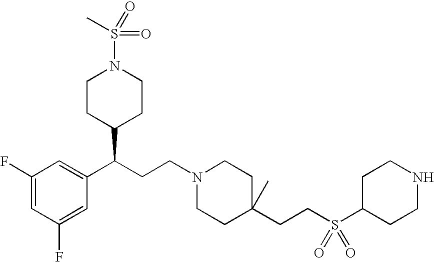

To a solution of benzyl 4-{[2-(1-{(3R)-3-(3,5-difluorophenyl)-3-[1-(methylsulfonyl)piperidin-4-yl]propyl}-4-methylpiperidin-4-yl)ethyl]sulfonyl}piperidine-1-carboxylate (Compound 1, Table VIII; 807 mg, 1.12 mmol) in ethanol (11 ml) was added palladium hydroxide 20% on carbon (78 mg, 0.112 mmol) and the system stirred under an atmosphere of hydrogen for 3 days. The mixture was filtered through celite, washed with ethanol and then the organics were concentrated in vacuo to give a yellow foam (590 mg); M+H 590.

### Step 2: Preparation of title compound

To a solution of 1-{(3R)-3-(3,5-difluorophenyl)-3-[1-(methylsulfonyl)piperidin-4-yl]propyl}-4-methyl-4-[2-(piperidin-4-ylsulfonyl)ethyl]piperidine (200 mg, 0.340 mmol) in DCM (3.5 ml) at 0° C. under a blanket of argon was added triethylamine (14011, 1.02 mmol) then methanesulfonyl chloride (54 μl, 0.680 mmol) and the reaction allowed to warm to ambient temperature and stirred for 5 hours. Further methanesulfonyl chloride (20111, 0.250 mmol) was added and the reaction stirred for a further 1 hour. The reaction was diluted with DCM (25 ml) and washed with saturated ammonium chloride solution (2×25 ml), brine (25 ml), dried over magnesium sulphate, filtered and concentrated in vacuo. Purification by flash chromatography using a gradient elution of 0 to 50% methanol in ethyl acetate gave a white foam (43 mg).

NMR (CDCl₃): 0.94 (s, 3H), 1.11-3.03 (m, 31H), 2.74 (s, 3H), 2.82 (s, 3H), 3.71 (m, 1H), 3.84 (m, 1H), 3.94 (m, 2H), 6.67 (m, 3H).

Additional NMR data: Compound No. 2 of Table I: NMR (CDCl₃): 1.6 (m, 4H), 2.0 (m, 2H), 2.3 (m, 6H), 2.6 (m, 2H) 3.1 (s, 3H), 3.2 (s, 3H), 3.4 (m, 2H), 4.2 (m, 1H), 6.7-6.8 (m, 3H), 7.5 (d, 2H), 7.9 (d, 2H), 8.2 (m, 4H).

Compound No. 4 of Table I: NMR (CDCl₃): 1.6-1.9 (m, 4H), 2.1 (m, 2H), 2.2-2.4 (m, 6H), 2.6-2.7 (m, 2H), 3.1 (s, 3H), 3.2 (s, 3H), 3.4 (m, 2H), 4.2 (t, 1H), 6.7-6.8 (m, 3H), 7.5 (d, 2H), 7.9 (d, 2H), 8.2 (m, 4H).

Compound No. 5 of Table I: NMR (CDCl₃): 0.7 (t, 3H), 1.2-1.5 (m, 6H), 1.6-1.8 (m, 5H), 2.2-2.4 (m, 5H), 3.0-3.1 (m, 5H), 3.15 (s, 3H), 4.1 (m, 1H), 6.6-6.8 (m, 3H), 7.4 (d, 2H), 7.85 (d, 2H), 8.1-8.2 (dd, 4H).

Compound No. 6 of Table I: NMR (400 MHz, CDCl₃) δ: 1.54-1.63 (m, 2H), 1.88 (d, 2H), 2.03-2.07 (m, 2H), 2.16-2.34 (m, 6H), 2.81-2.86 (m, 2H), 3.04 (s, 3H), 3.12 (s, 3H), 3.28-3.32 (m, 2H), 4.07 (t, 1H), 6.64-6.76 (m, 3H), 7.39 (d, 2H), 7.87 (d, 2H), 8.15 (d, 2H), 8.20 (d, 2H).

Compound No. 1 of Table II: NMR (CDCl₃): 0.8 (s, 3H), 1.1-1.4 (m, 5H), 1.5 (m, 1H), 1.6-1.7 (m, 5H), 1.9 (m, 3H), 2.0 (m, 3H), 2.2-2.3 (m, 3H), 2.4 (t, 1H), 2.5 (t, 1H), 2.6 (s, 3H), 3.0 (m, 5H), 3.6 (d, 1H), 3.7 (d, 1H), 7.0 (m, 2H), 7.1-7.2 (m, 3H), 8.0 (m, 4H).

Compound No. 2 of Table II: NMR (CDCl₃): 1.2-1.3 (m, 3H), 1.5-1.8 (m, 6H), 1.9-2.2 (m, 8H), 2.3 (m, 1H), 2.5 (m, 1H), 2.6 (m, 1H), 3.1 (s, 3H), 3.2-3.4 (m, 4H), 3.8 (m, 1H), 4.0 (m, 1H) 6.6 (m, 3H), 8.2 (m, 4H).

Compound No. 3 of Table II: NMR (CDCl₃): 1.3-1.8 (m, 10H), 2.0-2.3 (m, 8H), 2.4-2.7 (m, 4H), 2.8 (s, 3H), 3.2 (s, 3H), 3.3 (m, 2H), 3.8 (m, 1H), 3.9 (m, 1H), 6.7-6.8 (m, 3H), 8.2 (m, 4H).

Compound No. 4 of Table II: NMR (CDCl₃): 1.6-2.4 (m, 17H), 2.5-2.7 (m, 3H), 2.9-3.1 (m, 4H), 3.2 (s, 3H), 3.3 (m, 2H), 6.7-6.8 (m, 3H), 8.2 (m, 4H).

Compound No. 5 of Table II: NMR (CDCl₃): 0.9 (s, 3H), 1.2-1.5 (m, 4H), 1.6-1.8 (m, 7H), 1.9 (m, 1H), 2.0-2.5 (m, 8H), 3.1 (m, 2H), 3.2 (s, 3H), 3.3-3.5 (m, 2H), 3.9 (m, 1H), 4.1 (m, 1H), 6.7 (m, 3H), 8.2 (m, 4H).

Compound No. 6 of Table II: NMR (CDCl₃): 0.8 (s, 3H), 1.2-1.5 (m, 6H), 1.6-1.7 (m, 4H), 2.0-2.3 (m, 8H), 2.4 (m, 2H), 2.5-2.6 (m, 2H), 2.7 (s, 3H), 3.0-3.1 (m, 5H), 3.7 (m, 1H), 3.8 (m, 1H), 6.6-6.7 (m, 3H), 8.1-8.2 (m, 4H).

Compound No. 7 of Table II: NMR (CDCl₃): 1.2-1.7 (m, 8H), 1.9 (m, 2H), 2.0-2.6 (m, 12H), 2.7 (s, 3H), 3.1 (s, 3H), 3.3 (m, 2H), 3.7 (m, 1H), 3.8 (m, 1H), 6.6-6.7 (m, 3H), 8.2 (m, 4H).

Compound No. 8 of Table II: NMR (CDCl₃): 1.2-1.3 (m, 3H), 1.5-1.8 (m, 6H), 1.9 (m, 2H), 2.1-2.4 (m, 6H), 2.6-2.7 (m, 2H), 3.1 (s, 3H), 3.2-3.4 (m, 4H), 3.8 (m, 1H), 4.0 (m, 1H), 6.6-6.7 (m, 3H), 8.2 (m, 4H).

Compound No. 9 of Table II: NMR (CDCl₃): 0.78 (q, 1H), 0.82 (s, 3H), 1.08 (d, 3H), 1.19-1.40 (m, 6H), 1.53-1.70 (m, 5H), 1.77 (d, 1H), 1.95-2.21 (m, 5H), 2.28-2.40 (m, 3H), 3.07 (m, 1H), 3.11 (s, 3H), 3.28 (m, 1H), 3.41 (t, 1H), 4.00 (dd, 1H), 6.62 (m, 3H), 8.11 (d, 2H), 8.18 (d, 2H).

Compound No. 10 of Table II: NMR (CDCl₃): 0.75 (q, 1H), 1.09 (d, 3H), 1.19-1.30 (m, 2H), 1.55-1.82 (m, 8H), 1.94-2.19 (m, 6H), 2.27-2.43 (m, 2H), 2.48-2.63 (m, 1H), 2.71-2.90 (m, 1H), 3.10 (s, 3H), 3.25 (m, 2H), 3.40 (t, 1H), 4.00 (dd, 1H), 6.61 (m, 3H), 8.08-8.20 (m, 4H).

Compound No. 11 of Table II: NMR (CDCl₃): 0.8 (s, 3H), 1.3-1.4 (m, 4H), 1.6-2.2 (m, 12H), 2.25-2.4 (m, 3H), 2.5 (m, 1H), 3.8-3.1 (m, 6H), 3.15 (s, 3H), 6.6-6.7 (m, 3H), 8.1-8.2 (dd, 4H).

Compound No. 12 of Table II: NMR (CDCl₃): 0.7 (t, 3H), 1.2-1.8 (m, 14H), 1.9-2.1 (m, 3H), 2.2-2.4 (m, 5H), 2.5-2.65 (m, 2H), 2.75 (s, 3H), 3.0 (m, 2H), 3.1 (s, 3H), 3.7 (m, 1H), 3.8 (m, 1H), 6.6-6.7 (m, 3H), 8.1-8.2 (dd, 4H).

Compound No. 13 of Table II: NMR (CDCl₃): 1.2-1.8 (m, 9H), 1.9-2.0 (m, 2H), 2.05-2.3 (m, 6H), 2.4-2.75 (m, 5H), 2.8 (s, 3H), 3.1 (s, 3H), 3.2-3.25 (m, 5H), 3.8 (m, 1H), 3.9 (m, 1H), 6.6-6.8 (m, 3H), 8.2 (m, 4H).

Compound No. 14 of Table II: NMR (CDCl₃): 1.14-1.53 (m, 5H), 1.82-1.88 (m, 2H), 1.93-2.06 (m, 5H), 2.11-2.28 (m, 3H), 2.34-2.40 (m, 1H), 2.51 (t, 2H), 2.61 (t, 2H), 2.73 (s, 3H), 2.75-2.81 (m, 1H), 2.83-2.91 (m, 1H), 3.12 (s, 3H), 3.26-3.32 (m, 2H), 3.72 (d, 1H), 3.85 (d, 1H), 6.59-6.71 (m, 3H), 8.14 (d, 2H), 8.19 (d, 2H).

Compound No. 15 of Table II: NMR (CDCl₃): 1.25 (m, 2H), 1.4-1.55 (m, 2H), 1.6-1.85 (m, 6H), 1.9 (m, 2H), 2.1-2.3 (m, 5H), 2.35-2.6 (m, 3H), 3.1 (s, 3H), 3.2 (m, 5H), 3.3-3.5 (m, 2H), 3.9 (m, 1H), 4.0-4.1 (m, 1H), 6.7 (m, 3H), 8.2-8.3 (dd, 4H).

Compound No. 16 of Table II: NMR (CDCl₃): 1.19 (m, 1H), 1.31 (m, 11H), 1.46 (m, 3H), 1.67 (m, 3H), 1.83 (m, 2H), 2.01 (m, 4H), 2.18 (m, 3H), 2.37 (t, 1H), 2.55 (m, 4H), 2.73 (s, 3H), 3.00 (s, 3H), 3.04 (m, 2H), 3.71 (m, 1H), 3.83 (m, 1H), 3.89 (s, 3H), 6.64 (m, 3H), 7.02 (d, 2H), 7.82 (d, 2H).

Compound No. 17 of Table II: NMR (CDCl₃): 0.57-1.23 (m, 10H), 1.30 (m, 2H), 1.42 (m, 2H), 1.56 (m, 2H), 1.69-2.23 (m, 12H), 2.20 (s, 4H), 2.66 (m, 2H), 2.92 (s, 3H), 3.16 (m, 1H), 3.28 (m, 1H), 6.12 (m, 3H), 6.49 (d, 2H), 7.27 (d, 2H).

Compound No. 18 of Table II: NMR (CDCl₃): 0.84 (s, 3H), 1.39 (m, 4H), 1.66 (m, 2H), 1.89 (m, 1H), 2.07-2.56 (m, 12H), 2.73 (s, 3H), 3.07 (m, 2H), 3.11 (s, 3H), 3.17 (m, 3H), 3.40 (m, 1H), 7.18 (d, 2H), 7.29 (m, 3H), 8.11 (d, 2H), 8.16 (d, 2H).

Compound No. 19 of Table II: NMR (CDCl₃): 0.84 (s, 3H), 1.23-1.81 (m, 7H), 2.02-2.44 (m, 8H), 2.51 (m, 4H), 2.75 (s, 3H), 3.07 (m, 2H), 3.12 (s, 3H), 3.19 (m, 4H), 3.39 (m, 1H), 6.73 (m, 3H), 8.12 (d, 2H), 8.17 (d, 2H).

Compound No. 20 of Table II: NMR (CDCl₃): 1.02 (s, 3H), 1.23-3.31 (m, 22H), 2.78 (s, 3H), 3.47 (m, 3H), 3.92 (s, 3H), 6.79 (m, 3H), 7.06 (d, 2H), 7.83 (d, 2H).

Compound No. 21 of Table II: NMR (300 MHz, CDCl₃) δ: 0.86-0.94 (m, 1H), 1.01 (s, 3H), 1.42-1.62 (m, 8H), 1.67-1.77 (m, 1H), 1.83-2.27 (m, 7H), 2.43 (d, 1H), 2.70 (d, 1H), 2.83 (d, 1H), 3.12 (s, 3H), 3.28-3.34 (m, 2H), 3.52-3.63 (m, 1H), 3.69-3.84 (m, 2H), 6.66-6.71 (m, 3H), 8.15 (d, 2H), 8.21 (d, 2H).

Compound No. 1 of Table III: NMR (CDCl₃): 1.7-2.4 (m, 22H), 2.6-2.8 (m, 2H), 3.1 (s, 3H), 2.3 (m, 2H), 5.1 (m, 1H), 7.1-7.3 (m, 5H), 8.2 (m, 4H).

Compound No. 2 of Table III: NMR (DMSO): 1.4 (m, 4H), 1.5-1.8 (m, 10H), 2.0 (m, 2H), 2.2-2.4 (m, 7H), 3.3 (s, 3H), 3.4 (m, 2H), 4.3 (bs, 1H), 4.8 (m, 1H), 7.2-7.3 (m, 5H), 8.1-8.3 (m, 5H).

Compound No. 3 of Table III: NMR (CDCl₃): 1.0 (s, 3H), 1.5 (m, 4H), 1.7-1.9 (m, 8H), 2.1 (m, 2H), 2.2-2.3 (m, 6H), 2.4 (m, 2H), 2.6 (m, 1H), 3.1-3.2 (m, 5H), 5.2 (m, 1H), 7.2-7.4 (m, 5H), 7.9 (m, 1H), 8.2 (m, 4H).

Compound No. 1 of Table IV: NMR (d6 DMSO): 0.95-2.45 (m, 17H), 2.55-2.75 (m, 3H), 2.8 (s, 3H), 3.4-3.6 (m, 2H), 5.0 (s, 1H), 7.2-7.5 (m, 10H).

Compound No. 2 of Table IV: NMR (d6 DMSO): 0.95-2.45 (m, 17H), 2.55-2.75 (m, 3H), 2.8 (s, 3H), 3.4-3.6 (m, 2H), 5.0 (s, 1H), 7.1-7.5 (m, 9H).

Compound No. 1 of Table V: NMR (CDCl₃): 0.9 (t, 3H), 1.7 (m, 4H), 1.9 (q, 2H), 2.0 (m, 2H), 2.3 (m, 4H), 2.6 (m, 2H), 3.1 (s, 6H), 3.1 (s, 2H), 4.2 (m, 1H), 5.0 (s, 1H), 6.8 (m, 3H), 7.5 (dd, 4H), 8.0 (dd, 4H).

Compound No. 2 of Table V: NMR (CDCl₃): 1.35 (m, 5H), 1.4-1.8 (m, 6H), 2.0-2.35 (m, 10H), 2.4-2.75 (m, 5H), 2.8 (s, 3H), 3.15 (s, 3H), 3.45 (m, 2H), 3.8 (m, 1H), 3.85 (s, 2H), 3.9 (m, 1H), 6.7-6.8 (m, 3H), 7.55 (d, 2H), 7.95 (d, 2H).

Compound No. 1 of Table VI: NMR (CDCl₃): 1.7 (s, 3H), 2.1-2.3 (m, 6H), 2.4-2.6 (m, 6H), 3.05 (s, 3H), 3.1 (s, 3H), 4.15 (m, 1H), 6.6-6.8 (m, 3H), 7.4 (m, 2H), 7.75 (m, 4H), 8.2 (s, 1H), 8.4 (s, 11H).

Compound No. 1 of Table VII: NMR (CDCl₃): 1-1.8 (m, 101H), 2-2.6 (m, 10H), 2.7 (s, 2H), 2.75 (s, 3H), 3.6 (s, 3H), 3.7 (d, 1H), 3.9 (d, 1H), 6.6 (m, 3H), 6.8 (s, 1H), 6.9 (s, 1H).

Compound No. 2 of Table VII: NMR CDCl₃: 1.2-2.0 (m, 15H), 2.1-2.6 (m, 5H), 2.7 (s, 3H), 2.8 (s, 2H), 3.1 (s, 3H), 3.7-3.9 (m, 2H), 6.6 (m, 3H), 7.4-7.9 (q, 4H).

Compound No. 1 of Table VIII: NMR (CDCl₃): 0.90 (s, 3H), 1.13-1.84 (m, 101H), 1.93-2.55 (m, 15H), 2.61 (t, 2H), 2.74 (s, 3H), 2.83 (m, 3H), 3.00 (m, 1H), 3.72 (m, 1H), 3.84 (m, 1H), 4.37 (m, 2H), 5.13 (s, 2H), 6.66 (m, 3H), 7.34 (m, 5H).

Compound No. 2 of Table VIII: NMR (CDCl₃): 0.94 (s, 3H), 1.11-3.03 (m, 31H), 2.74 (s, 3H), 2.82 (s, 3H), 3.71 (m, 1H), 3.84 (m, 1H), 3.94 (m, 2H), 6.67 (m, 3H).

Compound No. 3 of Table VIII: NMR (CDCl₃): 1.07 (d, 3H), 1.14-1.65 (m, 6H), 1.98 (m, 7H), 2.17 (m, 1H), 2.34 (m, 1H), 2.41-2.85 (m, 10H), 2.77 (s, 3H), 3.03 (m, 1H), 3.13 (m, 1H), 3.40 (m, 4H), 3.74 (m, 1H), 3.86 (m, 1H), 4.14 (m, 2H), 6.74 (m, 3H).

Compound No. 1 of Table IX: NMR (CDCl₃): 6.75 (s, 1H), 6.7 (s, 1H), 6.6 (m, 3H), 3.8 (s, 3H), 3.65 (m, 1H), 3.2 (m, 2H), 2.7 (s, 3H), 2.5 (m, 4H), 2.15 (m, 4H), 1.95 (m, 3H), 1.8 (d, 4H), 1.65 (m, 2H), 1.5-1.2 (m, 4H).

Method A

### Preparation of (S)-3-phenyl-3-(4-methanesulfonylphenyl)propionaldehyde

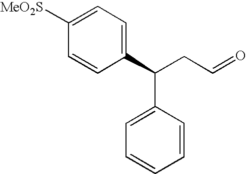

### Step 1: Preparation of E-(4S,5R)-1-(3-[4-methanesulphonylphenyl]acryloyl)-3,4-dimethyl-5-phenyl-imidazolidin-2-one

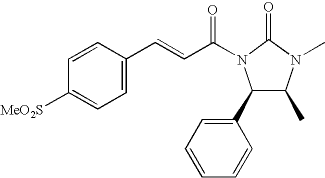

To a stirred solution of 3-(4-methanesulphonylphenyl)acrylic acid (7.14 g, 31.5 mmol) in DCM (10 mL) was added thionyl chloride (3 mL, 34.7 mmol) dropwise and the resulting mixture was stirred at room temperature for 18 h. To this solution was added DIPEA (5.04 mL, 28.9 mmol) dropwise at room temperature. The resulting solution was added to a stirred solution of (4R,5S)-1,5-dimethyl-4-phenyl-imidazolidin-2-one (5.0 g, 26.3 mmol) in DCM (20 mL) and DIPEA (4.58 mL, 26.9 mmol) and the resulting mixture stirred at room temperature for 4 h. The mixture was washed with water and brine, pre-absorbed onto a Bond Elut and eluted with a gradient of isohexane to ethyl acetate giving the sub-title compound as a solid (7.61 g, 73%); NMR (CDCl₃): 0.84 (d, 3H), 2.89 (s, 3H), 3.04 (s, 3H), 3.98 (m, 1H), 5.42 (d, 1H), 7.20 (m, 2H), 7.32 (m, 3H), 7.69 (d, 1H), 7.74 (d, 2H), 7.93 (d, 2H), 8.31 (d, 1H); MS: 399.

### Step 2: Preparation of (4S,5R)-1-[(S)-3-(4-methanesulfonyl-phenyl)-3-phenyl-propionyl]-3,4-dimethyl-5-phenyl-imidazolidin-2-one

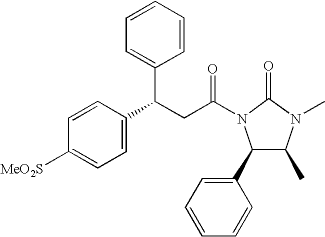

To a mixture of copper (I) iodide (960 mg, 5.0 mmol) and THF (20 mL) was added N,N,N′,N′-tetramethylethylenediamine (0.83 mL, 5.5 mmol) and the resulting mixture was stirred at room temperature for 10 min. then cooled to −78° C. Phenylmagnesium bromide (5.0 mL, 1M in THF, 5.0 mmol) was added and the resulting mixture stirred at −78° C. for 15 min. A solution of di-n-butylboron triflate (3.0 mL, IM in diethyl ether, 3.0 mmol) and (E)-(4S, 5R)-1-(3-[4-methanesulfonylphenyl]acryloyl)-3,4-dimethyl-5-phenyl-imidazolidin-2-one (1.0 g, 2.51 mmol) in THF (15 mL) was added and the resulting mixture was stirred whilst allowing to warm to room temperature for 18 h. The reaction mixture was washed with saturated aqueous ammonium chloride, water and brine, dried (MgSO₄) and evaporated. The residue was purified by eluting through a 20g Bond Elut with gradient of isohexane to ethyl acetate giving the sub-titled compound (1.49 g, 100%); NMR (CDCl₃): 0.78 (d, 3H), 2.82 (s, 3H), 3.00 (s, 3H), 3.78 (dd, 1H), 3.80 (m, 1H), 3.98 (dd, 1H), 4.72 (m, 1H), 5.19 (d, 1H), 6.99 (m, 2H), 7.22 (m, 8H), 7.48 (d, 2H), 7.79 (d, 2H); MS: 477.

### Step 3: Preparation of (S)-3-phenyl-3-(4-methanesulphonylphenyl)propan-1-ol

To a solution of (4S,5R)-1-[(S)-3-(4-methanesulphonyl-phenyl)-3-phenyl-propionyl]-3,4-dimethyl-5-phenyl-imidazolidin-2-one (846 mg, 1.78 mmol) in THF (20 mL) at 0° C. was added lithium aluminium hydride (3.6 mL, 1M in THF, 3.6 mmol) and the resulting mixture was stirred for 15 min. The reaction was quenched by the addition of 2M aqueous sodium hydroxide. The phases were separated and the organic phase pre-absorbed onto a Bond Elut and eluted with a gradient of isohexane to ethyl acetate giving the sub-titled compound as a white solid (285 mg, 55%); NMR (CDCl₃): 1.63 (br s, 1H), 2.33 (m, 2H), 3.00 (s, 3H), 3.59 (t, 2H), 4.28 (t, 1H), 7.23 (m, 5H), 7.43 (d, 2H), 7.82 (d, 2H).

### Step 4: Preparation of the Title Compound

To a solution of (S)-3-phenyl-3-(4-methanesulfonylphenyl)propan-1-ol (244 mg, 0.84 mmol) in DCM (5 mL) was added Dess-Martin periodinane (392 mg, 0.92 mmol) and the resulting mixture was stirred at room temperature for 1.5h. The mixture was washed with 2M aqueous sodium hydroxide (2×10 mL), dried and evaporated to give the title compound.

Method B

### (R)-3-(3,5-Difluorophenyl)-3-(4-methanesulfonylphenyl)propionaldehyde

This was prepared from (4S, SR)-1-(3-[4-methanesulfonylphenyl]acryloyl)-3,4-dimethyl-5-phenyl-imidazolidin-2-one and 3,5-difluorophenylmagnesium bromide using a method similar to that used to prepare (S)-3-phenyl-3-(4-methanesulfonyl-phenyl)propionaldehyde from phenylmagnesium bromide (Method A).

Method C

### Preparation of (3R)-3-(3,5-difluorophenyl)-3-[1-(methylsulfonyl)piperidin-4-yl]propanal

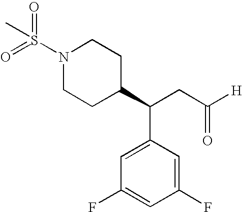

### Step 1 Preparation of (2E)-3-[1-(methylsulfonyl)piperidin-4-yl]acryloyl chloride

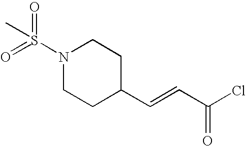

Oxalyl chloride (5.1 g) was added to a solution of (2E)-3-[1-(methylsulfonyl)piperidin-4-yl]acrylic acid (9.4 g) in dichloromethane containing 2-3 drops of DMF and the mixture was stirred at room temperature for 1.5 hours. The reaction mixture was evaporated to dryness and the residue obtained was used directly in the next step.

### Step 2 Preparation of (4R,5S)-1,5-dimethyl-3-{(2E)-3-[1-(methylsulfonyl)piperidin-4-yl]prop-2-enoyl}-4-phenylimidazolidin-2-one

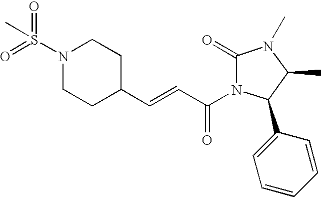

Lithium bis(trimethylsilyl)amide (8 ml of a 1M solution in THF) was added dropwise to a suspension of (4R,5S)-1,5-dimethyl-4-phenyl-2-imidazolidinone (1.52 g) in THF (20 ml) under argon at −10° C. The reaction mixture was stirred at −10° C. for 10 minutes, allowed to warm to 0° C. and maintained at this temperature for 10 minutes then cooled again to −101C. The solution of the acid chloride (2g dissolved in 10 ml of dichloromethane) prepared in Step 1 was added dropwise and the reaction mixture was allowed to warm to room temperature and washed with water (100 ml). The aqueous extract was extracted with ethyl acetate (3×50 ml) and the ethyl acetate extracts were dried and the residue passed through a 90g Biotage column eluting with a solvent gradient (50% ethyl acetate/isohexane-70% ethyl acetate/isohexane). Yield 1.89 g.

LC-MS MH⁺ 406.

NMR (CDCl₃): 0.8 (d, 3H), 1.5-1.6 (m, 3H), 1.9 (m, 2H), 2.3 (m, 1H), 2.7 (m, 2H), 2.75 (s, 3H), 2.8 (s, 3H), 3.75 (m, 2H), 3.9 (m, 1H), 5.3 (d, 1H), 6.85 (d-d, 1H), 7.1 (d, 1H), 7.2-7.35 (m, 3H), 7.45 (d, 1H).

### Step 3 Preparation of (4S,5R)-1-{(3R)-3-(3,5-difluorophenyl)-3-[1-(methylsulfonyl)piperidin-4-yl]propanoyl}-3,4-dimethyl-5-phenylimidazolidin-2-one

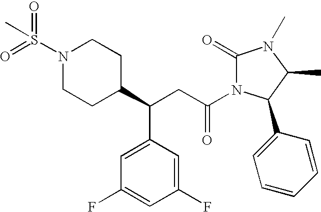 Step A

TMEDA (11.6 g) was added to a suspension of copper iodide (19.4 g) in THF (240 ml) under argon and the mixture was stirred for 45 minutes then cooled to −70° C. A solution of 3,5-difluorophenyl magnesium bromide in THF (201.1 ml of a 0.5M solution in THF) was added over 10 minutes and the mixture was stirred at −70° C. for 30 minutes.

Step B

Di-n-butylboron triflate (100.7 ml of 1M solution in dichloromethane) was added to a suspension of (4R,5S)-1,5-dimethyl-3-{(2E)-3-[1-(methylsulfonyl)piperidin-4-yl]prop-2-enoyl}-4-phenylimidazolidin-2-one (20.41 g) [Step 2] in THF maintained at −40C and stirring was continued for 10 minutes and the mixture was cooled to −70° C. and added via a cannula to the cuprate suspension prepared in step A. The reaction mixture was stirred at −70° C. for 1 hour and allowed to warm to room temperature, then saturated ammonium chloride solution (200 ml) was added. The THF was evaporated and ethyl acetate (200 ml) was added. Air was blown through this mixture for 1 hour. The ethyl acetate layer was collected and the aqueous portion was extracted with ethyl acetate (2×100 ml). The combined ethyl acetate extracts were washed with saturated ammonium chloride solution (2×100 ml), dried and evaporated to dryness. The residue was purified by chromatography on silica eluting with a solvent gradient of ethyl acetate-isohexane (1:1) to neat ethyl acetate to give the sub-title compound as a white solid, yield 25g.

NMR (CDCl₃): 0.78 (d, 3H), 1.2-1.6 (m, 6H), 1.9 (m, 1H), 2.4-2.65 (m, 2H), 2.75 (s, 3H), 2.85 (s, 3H), 3-3.2 (m, 2H), 3.7-3.9 (m, 4H), 5.2 (d, 1H), 6.6 (m, 3H), 6.85 (m, 2H), 7.2 (m, 3H).

### Step 4 Preparation of (3R)-3-(3,5-difluorophenyl)-3-[1-(methylsulfonyl)piperidin-4-yl]propan-1-ol

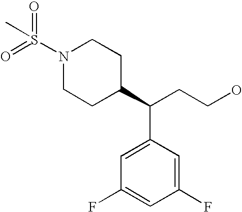

Lithium borohydride (48 ml of 2M solution in THF) was added to a solution of (4S,5R)-1-{(3R)-3-(3,5-difluorophenyl)-3-[1-(methylsulfonyl)piperidin-4-yl]propanoyl}-3,4-dimethyl-5-phenylimidazolidin-2-one (25g) in THF (200 ml) and the mixture was heated at 70° C. for 3 hours then allowed to cool to room temperature and stirring was continued for 16 hours. Ethanol was added carefully (20 ml) and the reaction mixture was acidified to pH 4 by addition of 2M HCl. The THF was evaporated and the residue dissolved in dichloromethane (100 ml) and this was washed with water (100 ml) and dried. The solvent was removed and the product was purified by chromatography on a Biotage 65 column eluted with a 1:1 mixture of ethyl acetate/isohexane. Yield 13g.

NMR (CDCl₃): 1.2-1.8 (m, 5H), 1.95-2.2 (m, 2H), 2.5-2.7 (m, 3H), 2.75 (s, 3H), 3.3-3.6 (m, 2H), 3.7-3.9 (m, 2H), 6.65 (m, 3H).

### Step 5 Preparation of Title Compound

Dess-Martin periodinane (1g) was added to a solution of (R) 3-(N-methanesulphonyl-piperidin-4-yl)-3-(3,5-difluorophenyl)propanol (0.8 g) in dichloromethane (40 ml) and the mixture was stirred for 1.5 hours. The reaction mixture was washed with 2M NaOH (2×20 ml) and dried. The solution of the title compound in dichloromethane was used in subsequent reactions.

Method D

### Preparation of (3R)-3-(3,5-difluorophenyl)-3-(tetrahydro-2H-pyran-4-yl)propanal

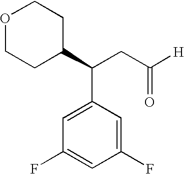

### Step 1. Preparation of (2E)-3-(tetrahydro-2H-pyran-4-yl)acrylic acid

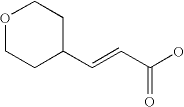

A mixture of tetrahydro-2H-pyran-4-carboxaldehyde (2.47 g), malonic acid (2.26 g) and piperidine (0.2 ml) in pyridine (15 ml) was heated to 100° C. for 4 hours. The reaction mixture was concentrated and partitioned between ethyl acetate (100 ml) and 1N HCl. The organic layer was dried and evaporated to give the sub-title compound, yield 2.77 g.

NMR CDCl₃: 1.4-1.8 (m, 4H), 2.4 (m, 1H), 3.4 (m, 2H), 4.0 (m, 2H), 5.8 (d, 1H), 7.0 (dd, 11H).

### Step 2: Preparation of (4R,5S)-1,5-dimethyl-4-phenyl-3-[(2E)-3-(tetrahydro-2H-pyran-4-yl)prop-2-enoyl]imidazolin-2-one

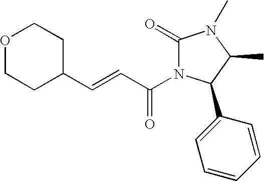 Step A To a solution of (2E)-3-(tetrahydro-2H-pyran-4-yl)acrylic acid (2.76 g) in anhydrous THF (25 ml) was added 1-chloro-N,N-2-trimethyl-1-propenylamine (2.31 ml) and the resulting mixture was stirred for 3 hours.
Step B To a suspension of (4R,5S)-1,5-dimethyl-4-phenyl-2-imidazidinone (3.32 g) in THF (25 ml), cooled to 5° C., was added dropwise lithium bis(trimethylsilyl)amide (19.2 ml of a 1M solution in THF) under argon. The reaction mixture was stirred for 30 minutes before the addition of the solution of the acid chloride from step A. The resulting mixture was stirred at room temperature for 18 hours. The reaction was quenched with 50% brine (100 ml) and extracted with ethyl acetate (3×100 ml) and the ethyl acetate extracts were dried and evaporated. The residue was recrystallised from ethanol to give the sub-title compound, yield 3.46 g.

NMR CDCl₃: 0.8 (d, 3H), 1.4-1.7 (m, 4H), 2.35 (m, 1H), 2.8 (s, 3H), 3.35 (m, 2H), 3.9 (m, 3H), 5.3 (d, 1H), 6.85 (dd, 1H), 7.1 (m, 2H), 7.25 (m, 3H), 7.4 (d, 1H).

### Step 3 Preparation of (4S,5R)-1-[(3R)-3-(3,5-difluorophenyl)-3-(tetrahydro-2H-pyran-4-yl)propanoyl]-3,4-dimethyl-5-phenylimidazolidin-2-one

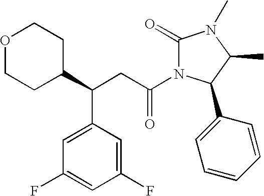

To a suspension of copper iodide (931 mg) in anhydrous THF (60 ml) under argon was added TMEDA (0.81 ml) and the resulting mixture was stirred for 20 minutes. The reaction mixture was cooled to −70° C. and 3,5-difluorophenyl magnesium bromide (9.8 ml of 0.5M solution in THF) was added dropwise and the mixture was stirred for a further 1 hour. A preformed solution of (4R,5S)-1,5-dimethyl-4-phenyl-3-[(2E)-3-(tetrahydro-2H-pyran-4-yl)prop-2-enoyl]imidazolin-2-one (800 mg) and dibutylboron triflate (2.93 ml of 1M solution in dichloromethane) in dichloromethane (2 ml) was added dropwise to the mixture. The reaction mixture was stirred for 1 hour at −70° C. and allowed to warm to room temperature, then saturated ammonium chloride (100 ml) and ethyl acetate (200 ml) was added. Air was blown through the mixture for 1 hour. The ethyl acetate was collected and the aqueous layer was extracted with ethyl acetate (2×100 ml). The combined ethyl acetate layers were washed with water, saturated EDTA, dried and evaporated to dryness. The residue was purified by chromatography on silica eluting with a solvent gradient of isohexane to 75% ethyl acetate/isohexane to give the sub-title compound as a solid. Yield 887 mg. M+H 443. NMR CDCl₃ 0.8 (d, 3H), 1.2-1.5 (m, 3H), 1.7 (m, 2H), 2.85 (s, 3H), 3.0 (m, 1H), 3.15-3.4 (m, 3H), 3.8-4.0 (m, 4H), 5.2 (d, 1H), 6.6-6.7 (m, 3H), 6.85 (m, 2H), 7.2 (m, 3H).

### Step 4 Preparation of (3R)-3-(3,5-difluorophenyl)-3-(tetrahydro-2H-pyran-4-yl)propan-1-ol

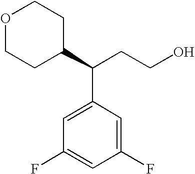

Lithium borohydride (1.5 ml of 2M solution in THF) was added to a solution of (4S,5R)-1-[(3R)-3-(3,5-difluorophenyl)-3-(tetrahydro-2H-pyran-4-yl)propanoyl]-3,4-dimethyl-5-phenylimidazolidin-2-one (882 mg) in anhydrous THF (20 ml) and the mixture was heated to 60° C. for 2 hours. The reaction mixture was cooled and quenched with saturated ammonium chloride and ethyl acetate and stirred for 20 minutes. The organic layer was dried and evaporated to dryness. The residue was purified by chromatography on silica eluting with a gradient of ethyl acetate and isohexane (10:90 to 50:50) to give the sub-title compound as an oil. Yield 345 mg. NMR CDCl₃: 1.2-1.4 (m, 2H), 1.6-1.85 (m, 4H), 2.15 (m, 1H), 2.5 (m, 1H), 3.25-3.6 (m, 4H), 3.9 (m, 1H), 4.05 (m, 1H), 6.7 (m, 3H).

### Step 5 Preparation of the title compound

Dess-Martin periodinane (628 mg) was added to a solution of (3R)-3-(3,5-difluorophenyl)-3-(tetrahydro-2H-pyran-4-yl)propan-1-ol (345 mg) in dichloromethane (10 ml) and the mixture was stirred for 2 hours. The reaction mixture was washed with 1N NaOH (10 ml) and dried. The solution of the title compound in dichloromethane was used in subsequent reactions.

In a similar manner but using 4-methyl-tetrahydro-pyran-4-carboxaldehyde (Method S) instead of tetrahydro-2H-pyran-4-carboxaldehyde in Step 1 was prepared (3R)-3-(3,5-difluorophenyl)-3-(4-methyltetrahydro-2H-pyran-4-yl)propanal. 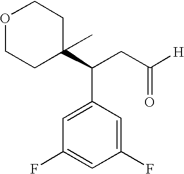
Method E

### Preparation of (3R)-3-(3,5-difluorophenyl)-3-[(2S)-2-methyltetrahydro-2H-pyran-4-yl]propanal

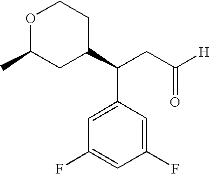

### Step 1 Preparation of (2S,4E/Z)-4-(methylmethylene)-2-methyltetrahydro-2H-pyran

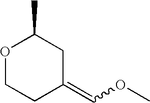

To a suspension of (methoxymethyl)triphenyl phosphine chloride (32g) in anhydrous THF (160 ml), cooled to −10° C., was added dropwise sodium bis(trimethylsilyl) amide (46.7 ml of 2M solution in THF). The reaction mixture was stirred for 1 hour and then a solution of (2S)-2-methyltetrahydro-4H-pyran-4-one (7.1g) in anhydrous THF (20 ml) was added over 5 minutes. The resulting mixture was allowed to warm to room temperature and stirred for 3 hours. The reaction was quenched with water (50 ml) and extracted with diethyl ether (3×100 ml). The organics were dried and evaporated to dryness. The resulting gum was treated with diethyl ether and filtered. The organics were evaporated to dryness and the resulting residue was purified by chromatography on silica eluting with ethyl acetate/isohexane (1:9) to give the sub-title compound (˜1:1 E/Z mixture of isomers) as an oil. Yield 6.22 g. NMR CDCl₃ 1.1 (dd, 3H), 1.45-2.1 (m, 3H), 2.4-2.55 (m, 1H), 3.2 (m, 2H), 3.4 (s, 3H), 3.85 (m, 1H), 5.7 (m, 1H).

### Step 2 Preparation of (2S)-2-methyltetrahydro-2H-pyran-4-carboxaldehyde

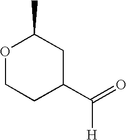

A mixture of (2S,4E/Z)-4-(methylmethylene)-2-methyltetrahydro-2H-pyran (6.22 g) and formic acid (40 ml, 88%) in water (20 ml) was heated, under argon, to 90° C. for 6 hours. The reaction mixture was cooled, neutralised with 6N sodium hydroxide and extracted with diethyl ether (3×150 ml). The organics were dried and evaporated to dryness. The residue was purified by chromatography on silica eluting with ethyl acetate/isohexane (3:7) to give the sub-title compound (4:1 mixture of cis/trans isomers) as an oil. Yield 4.065 g.

NMR CDCl₃: 1.25-1.4 (m, 4H), 1.5-2.2 (m, 3H), 2.45-2.7 (m, 1H), 3.4-3.5 (m, 2H), 3.85-4.1 (m, 1H), 9.65 (s, CHO cis), 9.8 (s, CHO trans).

### Step 3 Preparation of (2E)-3-[(2S)-2-methyltetrahydro-2H-pyran-4-yl]acrylic acid

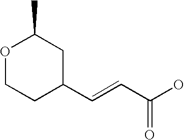

A mixture of (2S)-2-methyltetrahydro-2H-pyran-4-carboxaldehyde (4.0), malonic acid (6.495 g) and piperidine (0.1 ml) in pyridine (10 ml) was heated to 100° C. for 4 hours. The reaction mixture was concentrated and partitioned between ethyl acetate (100 ml) and 1N HCl. The organic layer was dried, evaporated and recrystallised form toluene to give the sub-title compound. Yield 2.48 g. NMR CDCl₃: 1.2 (m, 4H), 1.5 (m, 1H), 1.7 (m, 2H), 2.45 (m, 1H), 3.5 (m, 2H), 4.05 (m, 1H), 5.8 (d, 1H), 7.0 (dd, 1H).

### Step 4 Preparation of Title Compound

Using the method as described in Method D, steps 2-5, was prepared (3R)-3-(3,5-difluorophenyl)-3-[(2S)-2-methyltetrahydro-2H-pyran-4-yl] propanal.

In a similar manner, but starting from 2,6-dimethyltetrahydro-4H-pyran-2-one, was prepared (2E)-3-(2,6-dimethyltetrahydro-2H-pyran-4-yl)acrylic acid. NMR CDCl₃: 1.05 (m, 2H), 1.2 (m, 6H), 1.7 (m, 2H), 2.5 (m, 1H), 3.5 (m, 2H), 5.8 (d, 1H), 7.0 (dd, 1H). 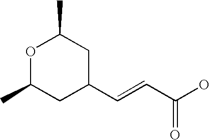
Method F

### Preparation of 3-Phenyl-3-(N-methanesulphonylpiperidin-4-yl)propionaldehyde

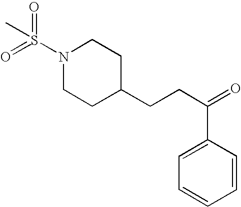

### Step 1: Preparation of 4-benzoyl-1-methanesulphonylpiperidine

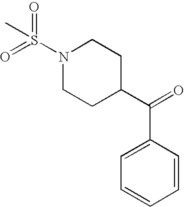

Methanesulphonyl chloride was added to a stirred slurry of 4-benzoylpiperidine hydrochloride (4.51 g) and triethylamine (8.35 ml) in dichloromethane (100 ml) at 0° C. The reaction mixture was allowed to warm to room temperature and was stirred for 16 hours. The mixture was diluted with dichloromethane (50 ml) and washed with ammonium chloride solution (2×25 ml) and brine (25 ml), dried and evaporated to dryness to give 4-benzoyl-1-methanesulphonylpiperidine as a white solid, yield 3.98 g. NMR (CDCl₃): 1.93 (m, 4H), 2.81 (s, 3H), 2.98 (dt, 2H), 3.40 (m, 1H), 3.77 (m, 2H), 7.43 (t, 2H), 7.57 (t, 1H), 7.89 (d, 2H).

### Step 2: Preparation of ethyl 3-phenyl-3-(N-methanesulphonylpiperidin-4-yl)acrylate

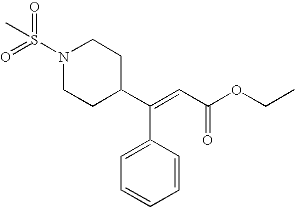

Lithium bis(trimethylsilyl)amide (16.3 ml of a 1M solution in THF) was added dropwise to a solution of triethylphosphonoacetate (2.93 ml) in THF at 0° C. under an argon atmosphere and the mixture was stirred for 30 minutes. A slurry of 4-benzoyl-1-methanesulphonylpiperidine (3.96 g) in THF (30 ml) was added, the reaction mixture was allowed to warm to room temperature and stirring was continued for 24 hours. The reaction mixture was diluted with dichloromethane (80 ml) and water (80 ml). The organic layer was washed with water and the combined aqueous extracts were in turn extracted with dichloromethane (50 ml). The combined dichloromethane extracts were washed with brine (25 ml), dried and evaporated to dryness. The residue was chromatographed on a 90g Biotage column eluted with a solvent gradient (30-75% ethyl acetate/isohexane) to give a less polar fraction (1.62 g) and a more polar fraction (0.53 g). Both fractions (cis/trans isomers) were combined and used for the next step.

Less polar NMR (CDCl₃): 1.27 (t, 3H), 1.69 (m, 2H), 1.81 (d, 2H), 2.72 (s, 3H), 2.72 (t, 2H), 3.81 (d, 2H), 3.88 (m, 1H), 4.21 (q, 2H), 5.78 (s, 1H), 7.11 (m, 2H), 7.27 (m, 3H).

More polar NMR (CDCl₃): 1.01 (t, 3H), 1.56 (m, 2H), 1.85 (d, 2H), 2.31 (m, 1H), 2.63 (t, 2H), 2.74 (s, 3H), 3.83 (d, 2H), 3.92 (q, 3H), 5.82 (s, 1H), 7.04 (d, 2H), 7.30 (m, 3H).

### Step 3: Preparation of ethyl 3-phenyl-3-(N-methanesulphonylpiperidin-4-yl)propionate

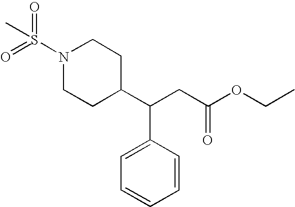

A solution of ethyl 3-phenyl-3-(N-methanesulphonylpiperidin-4-yl)acrylate (2.06 g) in ethanol (30 ml) was hydrogenated over 24 hours under a hydrogen filled balloon using 20% palladium hydroxide as catalyst. The reaction mixture was filtered through Celite® and the filtrate evaporated to dryness. The product obtained was used for the next step without further purification. MS: 340.

### Step 4: 3-Phenyl-3-(N-methanesulphonylpiperidin-4-yl)propan-1-ol

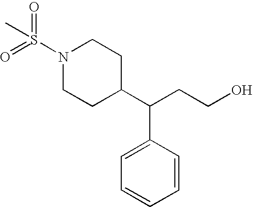

A solution of ethyl 3-phenyl-3-(N-methanesulphonylpiperidin-4-yl)propionate (2g) in THF (10 ml) was added to a suspension of lithium aluminium hydride (232 mg) in THF (20 ml) at 0° C. under argon over 30 minutes. The reaction mixture was allowed to warm to room temperature and stirred for 2 hours. Water (10 ml) was added followed by magnesium sulphate (10g). The reaction mixture was filtered and the filtrate evaporated to dryness to give the sub-titled product as a white foam, yield 1.57 g. NMR (CDCl₃): 1.40 (m, 4H), 1.57 (m, 1H), 1.78 (m, 1H), 2.01 (m, 2H), 2.45 (m, 2H), 2.58 (t, 1H), 2.70 (m, 3H), 3.31 (m, 1H), 3.42 (m, 1H), 3.67 (d, 1H), 3.80 (d, 1H), 7.04 (d, 1H), 7.19 (t, 1H), 7.29 (q, 2H).

### Step 5: Preparation of the title compound

Dess-Martin periodinane (739 mg) was added to a stirred solution of 3-phenyl-3-(N-methanesulphonylpiperidin-4-yl)propan-1-ol (454 mg) in dichloromethane (8 ml) and stirring was continued for 2 hours. The reaction mixture was diluted with dichloromethane (100 ml) and washed with 2M sodium hydroxide (2×50 ml), brine (50 ml) and dried. The product obtained on removal of the solvent was used in subsequent steps without purification.

Method G

### Preparation of 4,4-difluoro-N-[(1S)-3-oxo-1-phenylpropyl]cyclohexanecarboxamide

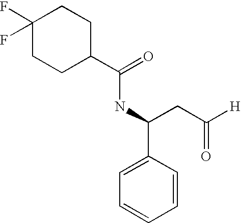

### Step 1: Preparation of 4,4-difluoro-N-[(1S)-3-hydroxy-1-phenylpropyl]cyclohexanecarboxamide

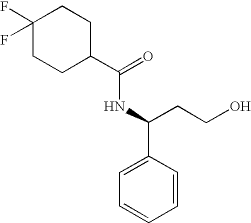

To a mixture of 4,4′-difluorocyclohexylcarboxylic acid (2.83 g) and HATU (6.56 g) in dimethyl formamide (15 ml) was added (S)-3-amino-3-phenylpropanol (2.37 g) and diisopropyl ethylamine (6.83 ml). The mixture was allowed to stir at room temperature for 6 days. The reaction mixture was poured into water (600 ml) and extracted with ethyl acetate (2×200 ml). The organics were washed with 1N NaOH (200 ml), brine (200 ml), dried (MgSO₄) and concentrated. The residue was purified by silica chromatography eluting with diethyl ether/iso-hexane to give the sub-title compound as a white solid. Yield 2.81 g. NMR (d6 DMSO): 1.66 (bm, 8H), 2.0 (m, 2H), 2.3 (m, 1H), 3.3 (m, 2H), 4.45 (t, 1H), 4.9 (m, 1H), 7.2 (m, 5H), 8.2 (m, 1H).

### Step 2: Preparation of title compound

In a similar manner to Method A, step 4, was prepared 4,4-difluoro-N-[(1S)-3-oxo-1-phenylpropyl]cyclohexanecarboxamide.

Method H

### Preparation of 4-methyl-4-(2-{[4-(methylsulfonyl)phenyl]sulfonyl}ethyl)piperidine

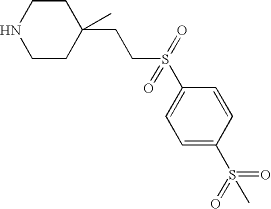

### Step 1: Preparation of tert-butyl 4-(1-cyano-2-ethoxy-2-oxoethylidene)piperidine-1-carboxylate

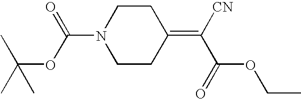

To a solution of tert-butyl 4-oxo-1-piperidinecarboxylate (20 g, 100.36 mmol) in toluene (150 ml) at room temperature was added ethyl cyanoacetate (10.64 ml, 100.36 mmol) followed by ammonium acetate (770 mg, 10.03 mmol) and acetic acid (0.57 ml, 10.03 mmol). The mixture was fitted with Dean Stark apparatus and stirred at reflux for 1 hour. The reaction was cooled to room temperature and evaporated to dryness and chromatographed (90g Silica Isolute, eluent 15% ethyl acetate/isohexane) to give white crystals (12.69 g, 43%); NMR (CDCl₃): 1.4 (t, 3H), 1.6 (s, 9H), 2.8 (t, 2H), 3.2 (t, 2H), 3.6 (t, 2H), 3.7 (t, 2H) 4.4 (q, 2H).

### Step 2: Preparation of tert-butyl 4-(1-cyano-2-ethoxy-2-oxoethyl)-4-methylpiperidine-1-carboxylate

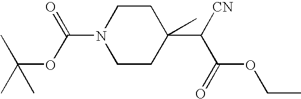

Anhydrous THF (350 ml) was added to copper cyanide (7.73 g, 86.32 mmol) under argon and cooled to −50° C. Methyl magnesium iodide (57.6 ml, of a 3M solution in diethyl ether) was added dropwise with caution over 20 minutes, ensuring temperature stayed below −40° C. The solution was stirred vigorously for 30 minutes and then allowed to come to room temperature over 1 hour. The solution was then re-cooled to −50° C. and tert-butyl 4-(1-cyano-2-ethoxy-2-oxoethylidene)piperidine-1-carboxylate (12.69 g, 43.16 mmol) in anhydrous THF (30 ml) was added and the mixture stirred for 1 hour at −50° C. before the temperature was allowed to come to room temperature. The reaction was quenched by the dropwise addition of saturated ammonium chloride. A further 100 ml of ammonium chloride was added followed by ethyl acetate (100 ml). The aqueous layer was then further extracted with ethyl acetate (3×50 ml). All organics were washed with water (2×50 ml), 1M HCl (1×75 ml), saturated sodium bicarbonate (1×75 ml) and finally brine (1×75 ml). The organics were dried (MgSO₄) and evaporated to give an orange/brown oil (13.31 g, 99%); NMR (CDCl₃): 1.3 (s, 3H), 1.4 (t, 3H), 1.5 (m, 11H), 1.7-1.8 (m, 2H), 3.2 (m, 2H), 3.5 (s, 1H), 3.8 (m, 2H), 4.4 (m, 2H).

### Step 3: Preparation of tert-butyl 4-(cyanomethyl)-4-methylpiperidine-1-carboxylate

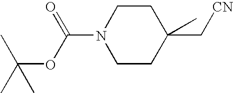

To a solution of tert-butyl 4-(1-cyano-2-ethoxy-2-oxoethyl)-4-methylpiperidine-1-carboxylate (13.3 g, 42.8 mmol) in DMSO (120 ml) and water (1.5 ml) was added lithium chloride (2.54 g) and the resulting mixture was heated to 160° C. for 2.5 hours. The reaction was cooled to room temperature and water (200 ml) was added. The mixture was extracted with diethyl ether (800 ml), washed with brine and dried. Evaporation under reduced pressure yielded a tan solid (8.77 g, 86%); NMR (CDCl₃): 1.1 (s, 3H), 1.4 (m, 13H), 2.2 (s, 2H), 3.1-3.2 (m, 2H), 3.5 (m, 2H).

### Step 4: Preparation of [1-(tert-butoxycarbonyl)-4-methylpiperidin-4-yl]acetic acid

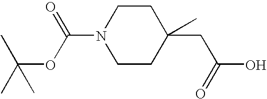

tert-Butyl 4-(cyanomethyl)-4-methylpiperidine-1-carboxylate (4.5 g, 18.9 mmol) was dissolved in concentrated hydrochloric acid (100 ml) and refluxed for 48 hours. The mixture was cooled diluted with water (200 ml) and then made basic to pH 12 with 2M NaOH. Di-tert-butyl dicarbonate (4.12 g, 18.9 mmol) was added and the mixture allowed to stir for 16 hours at room temperature. Solvent was evaporated and the solution was acidified to pH 5 with 2M HCl). The aqueous layer was extracted with dichloromethane (200 ml). The organic layer was dried and evaporated to yield a brown oil (3.54 g, 72%); NMR (CDCl₃): 1.2 (s, 3H), 1.5-1.7 (m, 13H), 2.4 (s, 2H), 3.4 (m, 2H), 3.6 (m, 2H).

### Step 5: Preparation of tert-butyl 4-(2-hydroxyethyl)-4-methylpiperidine-1-carboxylate

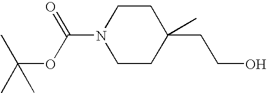

[1-(tert-Butoxycarbonyl)-4-methylpiperidin-4-yl]acetic acid (3.54 g, 13.77 mmol) was dissolved in anhydrous THF, under argon and cooled to −15° C. Borane:THF complex (13.8 ml of a 1M solution) was added and the reaction mixture was stirred for 1 h. The mixture was allowed to come to room temperature and slowly quenched with water (10 ml). Ethyl acetate (50 ml) was added followed by 2M sodium hydroxide (40 ml) and water (40 ml). The organic layer was separated and washed with brine (20 ml), dried and evaporated to give an orange oil (3g, 90%); NMR (CDCl₃): 0.9 (s, 3H), 1.2-1.3 (m, 4H), 1.4 (s, 9H), 1.7 (t, 3H), 3.2 (m, 2H), 3.4 (m, 2H), 3.6 (t, 3H).

### Step 6: Preparation of tert-butyl 4-methyl-4-(2-{[(4-methylphenyl)sulfonyl]oxy}ethyl)piperidine-1-carboxylate

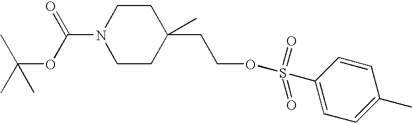

To a solution of tert-butyl 4-(2-hydroxyethyl)-4-methylpiperidine-1-carboxylate (3 g, 12.34 mmol) in dichloromethane (50 ml), cooled to 0° C., was added triethylamine (2.06 ml, 14.81 mmol) and p-toluene sulfonylchloride (2.59 g, 13.57 mmol). The reaction mixture was stirred for 20 h at room temperature. The mixture was washed with water (30 ml) and brine (30 ml). The organic layer was dried and evaporated. The crude oil was chromatographed (50g Silica Isolute, gradient elution, isohexane to 20% ethyl acetate/isohexane to give an oil (3.75 g, 77%); NMR (CDCl₃): 1.0 (s, 3H), 1.3-1.4 (m, 4H), 1.5 (s, 9H), 1.7 (t, 2H), 2.5 (s, 3H), 3.2-3.3 (m, 2H), 3.6 (m, 2H), 4.2 (t, 2H), 7.4 (d, 2H), 7.8 (d, 2H).

### Step 7: Preparation of tert-butyl 4-methyl-4-(2-{[4-(methylthio)phenyl]thio}ethyl)piperidine-1-carboxylate

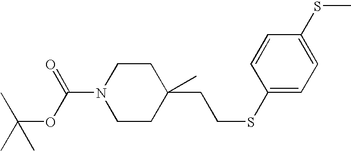

4-(Methylthio)benzenethiol (1476 mg, 9.45 mmol) was added to a suspension of sodium hydride (378 mg, 9.45 mmol, 60% dispersion in oil) in DMF (30 ml) at 0° C. The reaction mixture was stirred for 30 minutes at this temperature and then a solution of tert-butyl 4-methyl-4-(2-{[(4-methylphenyl)sulfonyl]oxy}ethyl)piperidine-1-carboxylate (3.75 g, 9.45 mmol) in DMF (10 ml) was added. Stirring continued for 16 h and after this time the mixture was evaporated, re-dissolved in DCM and washed with water and brine. The organic layer was dried and evaporated. The crude oil was chromatographed, (50g Silica Isolute, eluting 15% ethyl/isohexane) to give a clear oil (2.97 g, 82%); NMR (CDCl₃): 1.0 (s, 3H), 1.4 (m, 4H), 1.5 (s, 9H), 1.7 (m, 2H), 2.6 (s, 3H), 2.9 (m, 2H), 3.3 (m, 2H), 3.6 (m, 2H), 7.2-7.3 (m, 4H).

### Step 8: Preparation of tert-butyl 4-methyl-4-(2-{[4-(methylsulfonyl)phenyl]sulfonyl}ethyl)piperidine-1-carboxylate

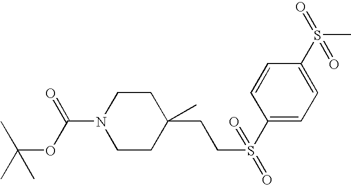

m-Chloroperbenzoic acid (7.7 g, 31.2 mmol, 70% purity) was added to a suspension of tert-butyl 4-methyl-4-(2-{[4-(methylthio)phenyl]thio}ethyl)piperidine-1-carboxylate (2.97 g, 7.8 mmol) in DCM (100 ml) at 0° C. The reaction was allowed to stir at room temperature for 3 hours. The mixture was washed with 2M NaOH (4×70 ml) and brine (1×70 ml). The organic layer was dried and evaporated to give a white solid (2.5 g, 72%); NMR (CDCl₃): 1.0 (s, 3H), 1.4 (m, 4H), 1.5 (s, 9H), 1.7 (s, 2H), 1.8 (m, 2H), 3.2 (m, 5H), 3.7 (m, 2H), 8.2 (m, 4H).

### Step 9: Preparation of 4-methyl-4-(2-{[4-(methylsulfonyl)phenyl]sulfonyl}ethyl)piperidine

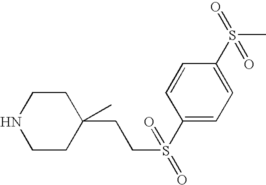

tert-Butyl 4-methyl-4-(2-{[4-(methylsulfonyl)phenyl]sulfonyl}ethyl)piperidine-1-carboxylate was dissolved in 4M HCl in dioxane. After stirring for 1 hour diethyl ether was added and the resulting white precipitate was filtered and washed with diethyl ether to give the title compound as a white solid (2.14 g, 100%), MH+ 346.3.

In a similar manner but using ethyl magnesium iodide in step 2 was prepared 4-ethyl-4-(2-{[4-(methylsulfonyl)phenyl]sulfonyl}ethyl)piperidine. 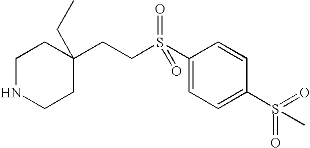
M+H 360

In a similar manner but using 4-mercaptotetrahydyopyran instead of 4-(methylthio)benzenethiol in Step 7 was prepared 4-methyl-4-[2-(tetrahydro-2H-pyran-4-ylsulfonyl)ethyl]piperidine

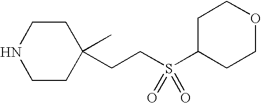

NMR (CDCl₃): 0.93 (s, 3H), 1.38 (m, 4H), 1.75 (m, 2H), 1.88 (m, 4H), 2.84 (m, 4H), 3.03 (m, 1H), 3.35 (m, 2H), 3.60 (m, 2H), 4.06 (d, 2H).

In a similar manner but using 4-methoxythiophenol in Step 7 was prepared 4-{2-[(4-methoxyphenyl)sulfonyl]ethyl}-4-methylpiperidine

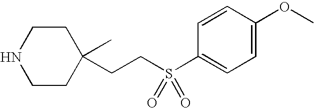

NMR (CDCl₃): 0.9 (s, 3H), 1.3 (m, 4H), 1.7 (m, 4H), 2.8 (m, 2H), 3.05 (m, 2H), 3.9 (s, 3H), 7.0 (d, 2H), 7.85 (d, 2H).

Method I

### Preparation of 4-(2-{[4-(methylsulfonyl)phenyl]sulfonyl}ethyl)piperidin-4-ol

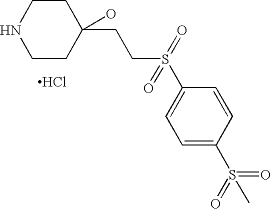

### Step 1: Preparation of tert-butyl 4-(2-ethoxy-2-oxoethyl)-4-hydroxypiperidine-1-carboxylate

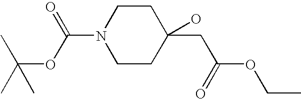

Ethyl bromoacetate (4.17 ml, 37.65 mmol) was added to a suspension of Rieke Zinc (4g, 37.65 mmol) in THF (60 ml), under argon, at such a rate to ensure only a small exotherm (room temperature to 35° C.) occurs. The mixture was allowed to cool to room temperature (10 minutes) and then tert-butyl 4-oxo-1-piperidinecarboxylate (5g, 25.1 mmol) in THF (15 ml) was added. After 3 hours stirring at room temperature the mixture was quenched by the slow, dropwise, addition of water (15 ml). A further 50 ml of water was added followed by ethyl acetate (50 ml) to give thick syrup. Brine (50 ml) was added and the mixture was extracted with ethyl acetate (×3), dried and evaporated to dryness. The residue was purified by chromatography (90g Silica Isolute, gradient elution, isohexane to 50% isohexane/ethyl acetate) to give an oil (3.43 g, 48%); NMR (CDCl₃): 1.4 (t, 3H), 1.5 (s, 9H), 1.6 (m, 2H), 1.7-1.8 (m, 2H), 2.5 (s, 2H), 3.3 (m, 2H), 3.6 (s, 1H), 3.9 (m, 2H), 4.3 (q, 2H).

### Step 2: Preparation of tert-butyl 4-hydroxy-4-(2-hydroxyethyl)piperidine-1-carboxylate

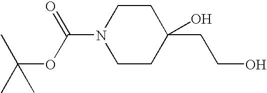

To a solution of tert-butyl 4-(2-ethoxy-2-oxoethyl)-4-hydroxypiperidine-1-carboxylate (3.43 g, 11.95 mmol) in anhydrous THF (40 ml) was added lithium aluminium hydride (12 ml of 1M solution in THF) under argon. After stirring at room temperature for 30 minutes ethyl acetate (20 ml) was added followed by water (0.3 ml), 2M NaOH (0.3 ml) and water (3 ml). After a few minutes celite (1g) was added and the mixture was filtered and evaporated to dryness to give the sub-title compound as an oil (2.93 g) which was used without further purification.

### Step 3: Preparation of tert-butyl 4-hydroxy-4-(2-{[(4-methylphenyl)sulfonyl]oxy}ethyl)piperidine-1-carboxylate

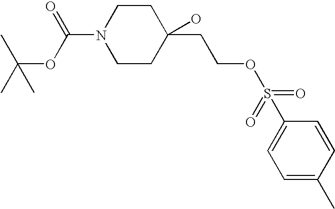

The sub-titled compound was made in a similar manner to Method H, step 6. Yield 38%. NMR (CDCl₃): 1.4 (s, 9H), 1.5 (m, 4H), 1.8 (m, 2H), 2.0 (s, 1H) 2.5 (s, 3H), 3.1 (m, 2H), 3.8 (m, 2H), 4.2 (m, 2H), 7.4 (d, 2H), 7.8 (d, 2H).

### Step 4: Preparation of tert-butyl 4-hydroxy-4-(2-{[4-(methylsulfonyl)phenyl]sulfonyl}ethyl)piperidine-1-carboxylate

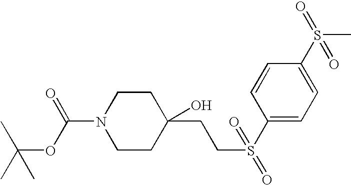

The sub-titled compound was prepared in a similar manner to Method H, steps 7-8 to give a solid. NMR (DMSO): 1.4 (s, 9H), 1.6 (m, 2H), 2.5 (m, 6H), 3.0 (m, 2H), 3.4 (m, 2H), 3.6 (m, 2H), 4.5 (s, 1H), 8.2 (m, 4H).

### Step 5: Preparation of title compound

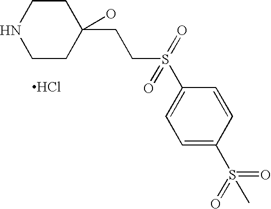

In a similar manner to Method H, step 9 was prepared 4-(2-{[4-(methylsulfonyl)-phenyl]sulfonyl}ethyl)piperidin-4-ol. MH⁺ 348.

In a similar manner but using 4-methoxythiophenol in Method I step 4 was prepared 4-(2-{[4-methoxyphenyl]sulfonyl}ethyl)piperidin-4-ol.

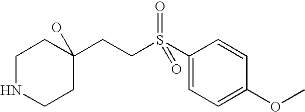

NMR (CDCl₃): 1.50 (m, 6H), 1.86 (m, 2H), 2.85 (m, 4H), 3.21 (m, 2H), 3.89 (s, 3H), 7.02 (d, 2H), 7.84 (d, 4H); M+H 300.

Method J

### Preparation of 4-fluoro-4-(2-{[4-(methylsulfonyl)phenyl]sulfonyl}ethyl)piperidine

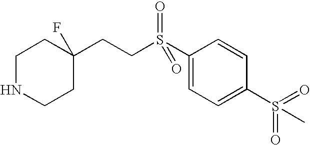

### Step 1: Preparation of tert-butyl 4-fluoro-4-(2-{[4-(methylsulfonyl)phenyl]sulfonyl}ethyl)piperidine-1-carboxylate

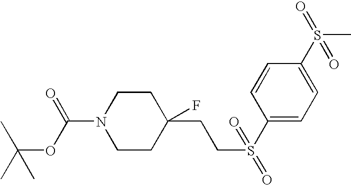

tert-Butyl 4-hydroxy-4-(2-{[4-(methylsulfonyl)phenyl]sulfonyl}ethyl)piperidine-1-carboxylate (Method H, step 5; 1.06 g, 2.37 mmol) in dichloromethane (25 ml) was added to a suspension of diethylaminosulfur trifluoride (0.63 ml, 4.74 ml) in dichloromethane (15 ml) at −70° C., under argon. The reaction was allowed to stir at this temperature for 90 minutes. The temperature was then allowed to increase to −10C with stirring for a further 30 minutes. The mixture was allowed to come to room temperature and saturated sodium bicarbonate (20 ml) was added. The organic layer was washed with further saturated sodium bicarbonate (3×20 ml) and then brine. The organic layer was dried and evaporated to give a yellow/white solid (1.05 g, 100%) MH + 350.2 (-Boc group).

### Step 2: Preparation of Title Compound

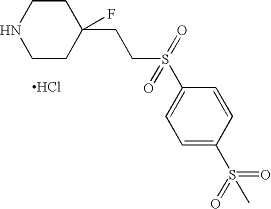

4-Fluoro-4-(2-{[4-(methylsulfonyl)phenyl]sulfonyl}ethyl)piperidine was prepared in a similar manner to 4-methyl-4-(2-{[4-(methylsulfonyl)phenyl]sulfonyl}ethyl)piperidine

(Method H, step 9) to give a white solid (837 mg, 100%) MH+ 350.15.

Method K

### Preparation of 4-methoxy-4-(2-{[4-(methylsulfonyl)phenyl]sulfonyl}ethyl)piperidine

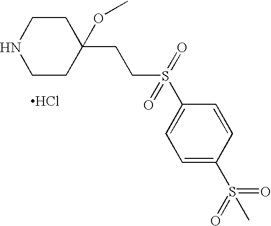

### Step 1: Preparation of tert-butyl 4-methoxy-4-(2-{[4-(methylsulfonyl)phenyl]sulfonyl}ethyl)piperidine-1-carboxylate

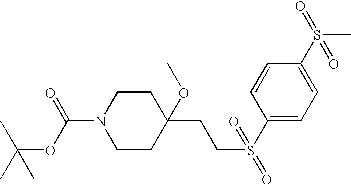

tert-Butyl 4-hydroxy-4-(2-{[4-(methylsulfonyl)phenyl]sulfonyl}ethyl)piperidine-1-carboxylate (Method H, step 5) (447 mg, 1 mmol) was added to a suspension of sodium hydride (40 mg, 1 mmol) in DMF (10 ml) at 0° C. and stirred at this temperature for 30 minutes and then methyl iodide (0.062 ml, 1 mmol) was added. After 2 hours, the reaction mixture was concentrated. The residue was dissolved in dichloromethane, washed with water, brine and then dried and evaporated to give a gum (460 mg, 100%) MH+ 362 (-Boc).

### Step 2: Preparation of Title Compound

4-Methoxy-4-(2-{[4-(methylsulfonyl)phenyl]sulfonyl}ethyl)piperidine was prepared in a similar manner to 4-methyl-4-(2-{[4-(methylsulfonyl)phenyl]sulfonyl}ethyl)piperidine (Method H, step 9) to give clear gum (250 mg, 63%).

Method L

### Preparation of N-(4-ethylpiperidin-4-yl)-2-[4-(methylsulfonyl)phenyl]acetamide

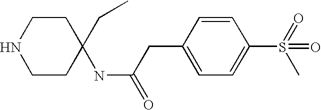

### Step 1: Preparation of tert-butyl4-amino-4-ethylpiperidine-1-carboxylate

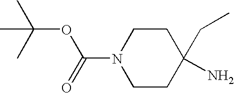

Step A. To a solution of 1-(tert-butoxycarbonyl)-4-ethylpiperidine-4-carboxylic acid (CAS188792-67-8) (6.72 g) in dry toluene (100 ml) was added DPPA (6.76 ml) followed by triethylamine (4.36 ml) and the resulting mixture was heated to 100° C. under an argon atmosphere for 1 hour. The reaction mixture was allowed to cool and washed with saturated sodium bicarbonate. The organic extracts was dried (MgSO₄), filtered and evaporated to dryness to give the intermediate isocynate (8.15 g) which was used without further purification.

Step B. To a solution of the above solid from step A (3.28 g) in THF (50 ml) was added potassium trimethylsilanolate (3.68 g) and the resulting mixture was stirred at room temperature for 18 hours. The reaction mixture was partitioned between dichloromethane and saturated sodium bicarbonate. The organic extracts were dried (MgSO₄) and evaporated to dryness to give the sub-title compound (2.42 g) as an orange oil which was used without further purification. NMR (d6 DMSO): 0.75 (t, 3H), 1.1-1.4 (m, 6H), 1.3 (s, 9H), 3.1 (m, 2H), 3.45 (m, 2H).

### Step 2: Preparation of tert-butyl 4-ethyl-4-({[4-(methylsulfonyl)benzyl]amino}carbonyl)piperidine-1-carboxylate

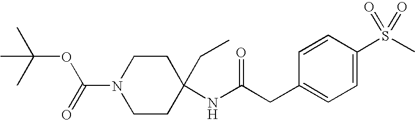

To a solution of [4-(methylsulfonyl)phenyl]acetic acid (395 mg) in dichloromethane (20 ml) was added disopropyldiethylamine (0.38 ml) followed by HATU (700 mg) and mixture was stirred for 10 minutes before the addition of tert-butyl4-amino-4-ethylpiperidine-1-carboxylate (420 mg). The resulting mixture was stirred at room temperature for 18 hours. The reaction mixture was partitioned between dichloromethane and water. The organic extracts were dried (MgSO₄) and evaporated to dryness. The residue was purified chromatography on silica eluting with gradient of ethyl acetate and isohexane to give the sub-title compound as an oil (750 mg). NMR (CDCl₃): 0.9 (t, 3H), 1.5 (s, 9H), 1.5 (m, 2H), 1.9 (m, 2H), 2.1 (m, 2H), 3.0 (m, 2H), 3.1 (s, 3H), 3.7 (s, 2H), 3.8 (m, 2H), 5.2 (s, 1H), 7.6 9d, 2H), 8.0 (d, 2H).

### Step 3: Preparation of Title Compound

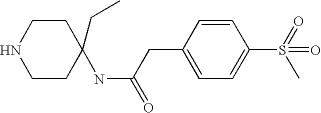

N-(4-Ethylpiperidin-4-yl)-2-[4-(methylsulfonyl)phenyl]acetamide was prepared in a similar manner to 4-methyl-4-(2-{[4-(methylsulfonyl)phenyl]sulfonyl}ethyl)piperidine (Method H, step 9) to give a gum. (MH⁺ 325).

Method M

### Preparation of 4-[(1-methyl-1H-imidazol-2-yl)methyl]piperidin-4-ol

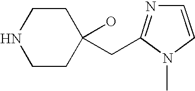

### Step 1: Preparation of tert-butyl 4-hydroxy-4-[(1-methyl-1H-imidazol-2-yl)methyl]piperidine-1-carboxylate

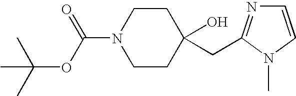

1,2-Dimethylimidazole (2.5 g) was dissolved in THF (100 ml) and cooled to −70° C. n-Butyl lithium (16.3 ml) was added drop wise. The reaction mixture was allowed to warm to −15° C. and stirred at −15° C. for 20 minutes. The reaction was cooled to −78° C. and tert-butyl 4-oxo-1-piperidine carboxylate added as solid. The reaction mixture was allowed to warm to room temperature and was evaporated to dryness. The residue was dissolved in dichloromethane (100 ml) and washed with saturated ammonium chloride (2×50 ml), dried over MgSO₄ and evaporated. The residue was purified by chromatography eluting with ethyl acetate to 40% methanol/ethyl acetate to yield tert-butyl 4-hydroxy-4-[(1-methyl-1H-imidazol-2-yl)methyl]piperidine-1-carboxylate as a gum (yield 400 mg; M+H 296); NMR CDCl₃: 1.2 (s, 3H), 1.45 (m, 2H), 1.6 (m, 2H), 2.7 (s, 2H), 3.2 (m, 2H), 4.45 (s, 3H), 4.8 (m, 4H), 6.8 (s, 1H), 6.9 (s, 1H).

### Step 2: Preparation of Title Compound

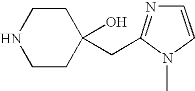

tert-Butyl 4-hydroxy-4-[(1-methyl-1H-imidazol-2-yl)methyl]piperidine-1-carboxylate (400 mg) was dissolved in TFA (10 ml) and stirred at room temperature for 1 hour. The TFA was evaporated to yield 300 mg of a gum, which was used without further purification.

Method N

### Preparation of 4-[4-(methylsulfonyl)benzyl]piperidin-4-ol

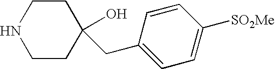

### Step 1: Preparation of tert-butyl 4-[4-(methylthio)benzylidene]piperidine-1-carboxylate

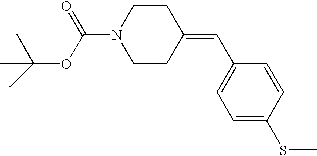

60% Sodium hydride in mineral oil (1.2 g) was suspended in DMF (100 ml) and cooled to 0° C. [4-(Methylthio)benzyl](triphenyl)phosphonium chloride (5.7 g) was added as solid over 10 minutes. The solution was stirred at 0° C. for 1 hour. tert-Butyl 4-oxo-1-piperidine carboxylate (2.5 g) was dissolved in DMF (20 ml) and added drop wise over 5 minutes. The reaction was allowed to warm to room temperature and stirred at this temperature for 4 hours. The solvent was evaporated and the residue dissolved in dichloromethane (50 ml) and washed with water (2×100 ml), dried over MgSO₄ and evaporated. The residue was purified by chromatography eluting with iso-hexane to 20% ethyl acetate/iso-hexane to yield 1.1 g of an off white solid. NMR CDCl₃: 1.4 (s, 9H), 2.3 (m, 2H), 2.4 (m, 2H), 2.45 (s, 3H), 3.4-3.5 (m, 4H), 6.3 (s, 1H), 7.1-7.3 (m, 4H).

### Step 2: Preparation of tert-butyl 2-[4-(methylsulfonyl)phenyl]-1-oxa-6-azaspiro[2.5]octane-6-carboxylate

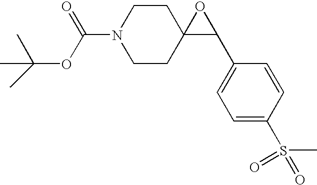

tert-Butyl 4-[4-(methylthio)benzylidene]piperidine-1-carboxylate (1.1 g) was dissolved in dichloromethane and 70% meta chloroperbenzoic acid (1.42 g) added. The reaction was incomplete after 1 hour so a further (1.4 g) of meta chloroperbenzoic acid was added. The reaction was stirred at room temperature for a further 2 hours and then washed with 2N NaOH (2×50 ml), dried over MgSO₄ and evaporated. The residue was purified by chromatography eluting with 10% ethyl acetate/iso-hexane to 40% ethyl acetate/iso-hexane to yield 1.1 g of an off white solid. NMR CDCl₃: 1.4 (s, 9H), 1.5-1.9 (m, 4H), 3.05 (s, 3H), 3.6-3.8 (m, 4H), 4.0 (s, 1H), 7.5 (d, 2H), 7.9 (d, 2H).

### Step 3: Preparation of title compound

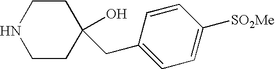

tert-Butyl 2-[4-(methylsulfonyl)phenyl]-1-oxa-6-azaspiro[2.5]octane-6-carboxylate (1.1 g) was dissolved in TFA (10 ml) and stirred at room temperature for 1 hour. The TFA was evaporated and methanol (100 ml) added to the residue and evaporated. The residue was dissolved in methanol (10 ml) and poured onto a 10g SCX2 cartridge and eluted with methanol (6×20 ml) and 1M ammonia/methanol (6×20 ml). The combined ammonia washings were evaporated to yield 400 mg of a white foam. NMR DMSOD6: 1.3-1.4 (m, 3H), 2.6-2.8 (m, 5H), 3.1 (s, 3H), 7.4-7.8 (q, 4H); M+H 270.

Method O

### Preparation of (3R)-3-(3,5-difluorophenyl)-3-(1,1-dioxidotetrahydro-2H-thiopyran-4-yl)propanal

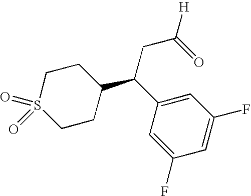

Prepared in a similar manner to that described in Method D except an additional oxidation step was inserted after step 3 as illustrated below:

### Preparation of (4S,5R)-1-[(3R)-3-(3,5-difluorophenyl)-3-(1,1-dioxidotetrahydro-2H-thiopyran-4-yl)propanoyl]-3,4-dimethyl-5-phenylimidazolidin-2-one

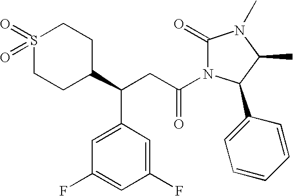

To a solution of (4S,5R)-1-[(3R)-3-(3,5-difluorophenyl)-3-(tetrahydro-2H-thiopyran-4-yl)propanoyl]-3,4-dimethyl-5-phenylimidazolidin-2-one (7.77 g) in dichloromethane (100 ml) was added meta-chloroperbenzoic acid (70% purity, 8.36 g) and the resulting mixture was stirred at room temperature for 18 hours. A further amount of meta-chloroperbenzoic acid (8.36 g) was added and the mixture was stirred for 18 hours. The organics were washed with 2N NaOH (6×30 ml), dried and evaporated to a yellow foam (4.34 g) which was used in the next step without further purification. NMR (CDCl₃): 0.9 (d, 3H), 1.7 (m, 2H), 1.9 (m, 3H), 2.1 (m, 1H), 2.8 (s, 3H), 2.85-3.1 (m, 3H), 3.2 (m, 2H), 3.7-3.9 (m, 2H), 5.2 (d, 1H), 6.6 (m, 3H), 6.85 (m, 2H), 7.2 (m, 2H); M+H 491.

Method P

### Preparation of (3-endo)-3-(1-methyl-H-imidazol-2-yl)-8-azabicyclo[3.2.1]octan-3-ol

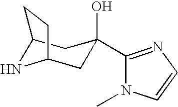

### Step 1: Preparation of 8-benzylbicyclo[3.2.1.]octan-3-one

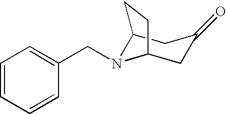

A solution of 2,5-dimethoxytetrahydrofuran (22.2 ml) in 0.1M HCl was refluxed for 1 hour and then cooled to 0° C. 1.3-Acetonedicarboxylic acid (25g), benzylamine (15.6 ml) and 10% sodium acetate (95 ml) was added in one portion and the resulting mixture was stirred at room temperature for 1 hour and then heated to 50° C. for 5 hours. The reaction mixture was cooled, basified with 2M sodium hydroxide, extracted with dichloromethane and washed with water. The organics were extracted with 1M hydrochloric acid and washed with dichloromethane. The aqueous layer was basified with 2M sodium hydroxide and extracted with ethyl acetate (3×100ml). The organic extracts were dried and evaporated to dryness to give the sub-title compound as a brown oil which was used without further purification (yield 13.66 g, MS 216 MH⁺).

### Step 2: Preparation of 8-benzyl-(3-endo)-(1-methyl-1H-imidazol-2-yl)-8-azabicyclo[3.2.1]octan-3-ol

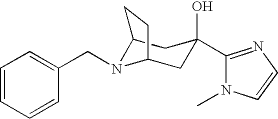

1-Methylimidazole (0.385 g) was dissolved in dry THF (20 ml) under an argon atmosphere and cooled to −78° C. A 1.6M solution of butyl lithium in hexane (3.125 ml) was added slowly and the resulting mixture was stirred at −78° C. for 90 minutes. A solution of 8-benzyl-8-azabicyclo[3.2.1]octan-3-one (1.05 g) in dry THF (5 ml) was added. The mixture was allowed to reach ambient temperature and stirred overnight. The reaction was quenched with saturated ammonium chloride solution and extracted with diethyl ether. The organic phase was dried over MgSO₄, filtered and evaporated to an oil, which was purified by column chromatography using a 40g SCX column eluting with CH₂Cl₂/CH₃OH/0.880 ammonia (9/1/0.1) to yield 8-benzyl-(3-endo)-(1-methyl-1H-imidazol-2-yl)-8-azabicyclo[3.2.1]octan-3-ol as a white solid. Yield 342 mg.

NMR (CDCl₃): 7.4 (m, 2H), 7.25 (m, 3H), 6.85 (s, 1H), 6.8 (s, 1H), 3.85 (s, 3H), 3.6 (s, 2H), 3.3 (s, 2H), 2.55 (m, 2H), 2.25 (m, 2H), 2.05 (m, 2H), 1.85 (m, 2H); MH+ 298.34.

### Step 3: Preparation of Title Compound

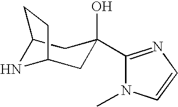

Benzyl-(3-endo)-(1-methyl-1H-imidazol-2-yl)-8-azabicyclo[3.2.1]octan-3-ol (0.32 mg) was dissolved in ethanol (30 ml). Ammonium formate (0.63 g) and 10% Pd/C catalyst (0.032 g) was added and the resulting mixture was heated at reflux for 18 hours. The reaction mixture was filtered and evaporated to yield (3-endo)-3-(1-methyl-1H-imidazol-2-yl)-8-azabicyclo[3.2.1]octan-3-ol as a solid. Yield 0.215 g.

NMR (CDCl₃): 6.85 (s, 1H), 6.8 (s, 1H), 3.85 (s, 3H), 3.6 (s, 2H), 2.45 (m, 2H), 2.3 (m, 2H), 1.95 (m, 2H), 1.89 (m, 2H); MH+ 208.32.

Method Q

### Preparation of N-ethyl-N-(4-methylpiperidin-4-yl)-2-[4-(methylsulfonyl)piperidin-1-yl]acetamide

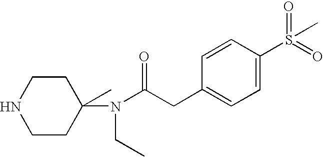

### Step 1: Preparation of tert-butyl4-amino-4-methylpiperidine-1-carboxylate

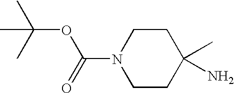 Step A. To a solution of 1-(tert-butoxycarbonyl)-4-ethylpiperidine-4-carboxylic acid (CAS 188792-67-8) (1.71 g) in dry toluene (30 ml) was added DPPA (1.82 ml) followed by triethylamine (1.17 ml) and the resulting mixture was heated to 100° C. under an argon atmosphere for 1.5 hour. The reaction mixture was allowed to cool and washed with saturated sodium bicarbonate. The organic extracts was dried (MgSO₄), filtered and evaporated to dryness to give the intermediate isocyanate (1.69 g), which was used without further purification.

Step B. To a solution of the above solid from step A (1.69 g) in THF (30 ml) was added potassium trimethylsilanolate (2g) and the resulting mixture was stirred at room temperature for 18 hours. The reaction mixture was partitioned between dichloromethane and saturated sodium bicarbonate. The organic extracts were dried (MgSO₄) and evaporated to dryness to give the sub-title compound (1.21 g) as an orange oil which was used without further purification.

NMR (CDCl₃): 1.2 (s, 3H), 1.4-1.7 (m, 13H), 3.4-3.6 (m, 4H).

### Step 2: Preparation of tert-butyl 4-(ethylamino)-4-methylpiperidine-1-carboxylate

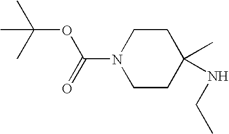

To a solution of tert-butyl4-amino-4-methylpiperidine-1-carboxylate (1.21 g) at 0° C. was added acetaldehyde (0.32 ml) and then stirred at this temperature for 1 hour. After this time sodium triacetoxyborohydride (1.44 g) was added and the resulting mixture was stirred at room temperature for 18 hours. The reaction mixture was partitioned between dichloromethane and saturated sodium bicarbonate. The organic extracts were dried (MgSO₄) and evaporated to dryness to give the sub-title compound (1.23 g) as an oil which was used without further purification.

NMR (CDCl₃): 1.2 (m, 6H), 1.4-1.65 (m, 13H), 2.7 (q, 2H), 3.3-3.7 (m, 4H).

### Step 3: Preparation of tert-butyl 4-(ethyl{[4-(methylsulfonyl)phenyl]acetyl}amino)-4-methylpiperidine-1-carboxylate

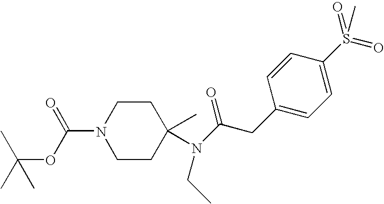

To a solution of 4-methylsulfonyl phenylacetic acid (1.49 g) in dichloromethane (10 ml) was added oxalyl chloride (0.66 ml) then a catalytic amount of dimethylformamide. The resulting mixture was stirred at room temperature for 2 hours. After this time the mixture was evaporated to dryness and then redissolved in dichloromethane. This was added to solution of tert-butyl 4-(ethylamino)-4-methylpiperidine-1-carboxylate (0.84 mg) in dichloromethane (10 ml). The resulting mixture was stirred at 60° C. for 2 hours and then at room temperature for 18 hours. The reaction mixture was partitioned between dichloromethane water. The organic extracts were dried (MgSO₄) and evaporated to dryness. The crude mixture was purified on silica using a gradient elution 1:1 Ethyl acetate:Hexane to Ethyl acetate to yield tert-butyl 4-(ethyl{[4-(methylsulfonyl)phenyl]acetyl}amino)-4-methylpiperidine-1-carboxylate 0.45 g.

NMR (CDCl₃): 1.4 (t, 3H), 1.55 (s, 9H), 1.6 (s, 3H), 2.1 (m, 4H), 3.15 (s, 3H), 3.2 (m, 2H), 3.45 (m, 2H), 3.75 (m, 2H), 3.85 (s, 2H), 7.5 (d, 2H), 8.0 (d, 2H).

### Step 4: Preparation of title compound

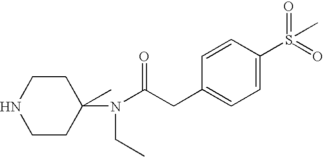

tert-Butyl 4-(ethyl {[4-(methylsulfonyl)phenyl]acetyl} amino)-4-methylpiperidine-1-carboxylate (0.43 g) was dissolved in dichloromethane (15 ml) to which was added trifluoroacetic acid (5 ml). The resulting mixture was stirred for 1.5 hours at room temperature. The crude mixture was evaporated to dryness and then partitioned between dichloromethane and 2M sodium hydroxide solution. The organic extracts were dried over MgSO₄ and evaporated to dryness to give the title compound as an orange foam (0.32 g), M+H 339.

Method R

### Preparation of 1-(4-methylpiperidin-4-yl)-5-(methylsulfonyl)-1H-benzimidazole

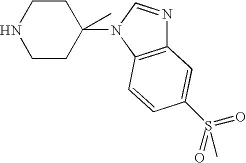

### Step 1: Preparation of tert-butyl 4-methyl-4-{[4-(methylsulfonyl)-2-nitrophenyl]amino}piperidine-1-carboxylate

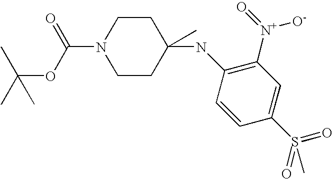

To a solution of tert-butyl4-aminopiperidine-1-carboxylate (1.5 g) in dimethyl sulphoxide (20 ml) was added 2-fluoro-5-methylsulfonylnitrobenzene (1.53 g) followed by anhydrous potassium carbonate (3.4 g) and the resulting mixture was heated to 100° C. for 3 hours. The mixture was cooled, quenched with water (100 ml) and extracted with ethyl acetate (×3). The organics were dried and evaporated to dryness to give the sub-title compound, which was used without further purification. Yield 2.43 g.

NMR (CDCl₃): 1.45 (s, 9H), 1.6 (s, 3H), 1.8 (m, 2H) 2.1 (m, 2H) 3.0 (s, 3H) 3.2 (m, 2H) 3.8 (m, 2H) 7.15 (d, 2H) 7.8 (d, 2H) 7.8 (m, 2H).

### Step 2: Preparation of tert-butyl 4-methyl-4-[5-(methylsulfonyl)-1H-benzimidazol-1-yl]piperidine-1-carboxylate

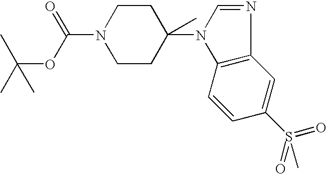

To a solution of tert-butyl 4-methyl-4-[5-(methylsulfonyl)-1H-benzimidazol-1-yl]piperidine-1-carboxylate (2.43 g) in ethanol/acetic acid (100 ml) was added triethyl orthoformate (9.7 ml) followed by a catalytic amount of 10% palladium on carbon. The resulting mixture was placed under a hydrogen atmosphere (3 bar) and heated to 80° C. for 16 hours. The mixture was cooled, filtered and evaporated to dryness to give a dark green foam. Yield 2.31 g.

NMR (CDCl₃): 1.45 (s, 9H), 1.75 (s, 3H), 2.2 (m, 2H), 2.4 (m, 2H), 3.1 (s, 3H), 3.4 (m, 2H), 3.7 (m, 2H), 7.7 (m, 1H), 7.8 (m, 1H), 8.2 (m, 1H), 8.4 (m, 1H).

### Step 3: Preparation of: Title Compound

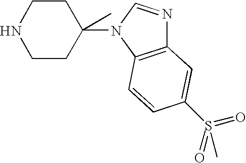

tert-Butyl 4-methyl-4-[5-(methylsulfonyl)-1H-benzimidazol-1-yl]piperidine-1-carboxylate (2.31 g) was dissolved in dichloromethane (15 ml) to which was added trifluoroacetic acid (5 ml). The resulting mixture was stirred for 1.5 hours at room temperature. The crude mixture was evaporated to dryness and then partitioned between dichloromethane and 2M sodium hydroxide solution. The organic extracts were dried over MgSO₄ and evaporated to dryness to give the title compound as an orange foam (1.17 g). M+H 294.

Method S

Preparation of 4-methyl-tetrahydro-pyran-4-carboxaldehyde 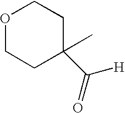
Step 1: Preparation of 4-methyl-tetrahydro-pyran-4-carboxylic acid methyl ester

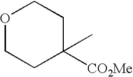

Tetrahydropyran-4-carboxylic acid methyl ester (14.42 g) was dissolved in anhydrous tetrahydrofuran (250 ml) and cooled to −78° C. under an atmosphere of argon. To this stirred solution was added, via syringe, lithium bis(trimethylsilyl)amide (1M solution in THF, 100 ml). The solution was allowed to warm to 0° C., stirred for 15 minutes, then cooled to −78° C. To the cooled solution was added, dropwise via syringe, iodomethane (6.2 ml). The solution was stirred for 30 minutes then allowed to warm slowly to room temperature and stirred for a further 3 hours. The reaction was then quenched with saturated aqueous ammonium chloride and partitioned with ethyl acetate. The aqueous portions were further extracted with ethyl acetate then the combined organic fractions were washed with water then brine then dried (MgSO₄) and filtered. Evaporation of solvents under reduced pressure gave a yellow oil which was purified by two successive rounds of column chromatography using a gradient of ethyl acetate in iso-hexane to give the sub-titled compound (7.25 g) as a yellow oil.

NMR (CDCl₃): 1.23 (s, 3H), 1.49 (t, 2H), 2.02-2.10 (m, 2H), 3.43-3.51 (m, 2H), 3.71 (s, 3H), 3.75-3.82 (m, 2H).

### Step 2: Preparation of (4-Methyl-tetrahydro-pyran-4-yl)-methanol

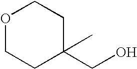

To a solution of 4-methyl-tetrahydro-pyran-4-carboxylic acid methyl ester (7.75 g) in anhydrous dichloromethane cooled to −78° C. was added, over 15 minutes via syringe, di-iso-butylaluminium hydride (1M solution in DCM, 123 ml). The reaction solution was left to stir at −78° C. for 3 hours then warmed to room temperature and left to stir for a further 2 hours. The reaction was then quenched with saturated ammonium chloride and partitioned with dichloromethane. The aqueous portions were further extracted with dichloromethane then the combined organic fractions were washed with brine, dried (MgSO₄) and evaporated to give a clear oil which was purified by column chromatography using a gradient of ethyl acetate in iso-hexane as eluent to give the sub-titled compound (5.54 g) as a clear oil.

NMR (CDCl₃): 1.02 (s, 3H), 1.25-1.21 (m, 2H), 1.58 (ddd, 2H), 2.60 (s, 1H), 3.37 (s, 2H), 3.62 (ddd, 2H), 3.74 (dt, 2H).

### Step 3: Preparation of 4-Methyl-tetrahydro-pyran-4-carboxaldehyde

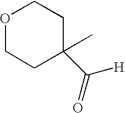

Pyridinium chlorochromate (11.55 g) and Celite® (23g) were mixed together and suspended in dichloromethane (250 ml) at 0° C. A solution of (4-methyl-tetrahydro-pyran-4-yl)-methanol (4.65 g) in dichloromethane (100 ml) was added to the stirred suspension and the reaction left to stir for 24 hours. The reaction was diluted with diethyl ether and filtered under suction, washing the filter cake with diethyl ether, to give, after evaporation of solvents under reduced pressure, a brown gum which was purified by column chromatography using a gradient of ethyl acetate in iso-hexane to give the product (3.26 g) as a clear oil.

NMR (CDCl₃): 1.11 (s, 3H), 1.50 (ddd, 2H), 1.94 (dt, 2H), 3.51 (ddd, 2H), 3.77 (dt, 2H), 9.47 (s, 1H).

Method T

Preparation of benzyl 4-{[2-(4-methylpiperidin-4-yl)ethyl]sulfonyl}piperidine-1-carboxylate

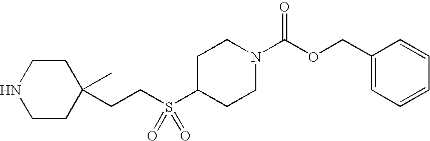

### Step 1: Preparation of tert-butyl 4-[2-({1-[(benzyloxy)carbonyl]piperidin-4-yl}sulfonyl)ethyl]-4-methylpiperidine-1-carboxylate

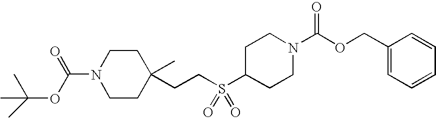

To a stirred slurry of 60% sodium hydride in mineral oil (220 mg, 5.5 mmol) in DMF (10 ml) at 0° C. under a blanket of argon was added a solution of benzyl 4-mercaptopiperidine-1-carboxylate (1.26 g, 5.02 mmol) in DMF (10 ml). The mixture was allowed to warm to ambient temperature for 30 minutes and then tert-butyl 4-methyl-4-(2-{[(4-methylphenyl)sulfonyl]oxy}ethyl)piperidine-1-carboxylate (Method H, step 6; 5.02 mmol) in DMF (5 ml) was added and the mixture stirred for 4 hours and then concentrated in vacuo. The residue was partitioned with DCM/water (100 ml/100 ml) and the organic layer separated and washed with brine (50 ml), dried over magnesium sulfate, filtered and concentrated in vacuo. The residue was dissolved in DCM (25 ml) and cooled to 0° C. and 70-75% strength 3-chloroperoxy benzoic acid (2.48 g, 10.1 mmol) was added. The mixture was allowed to warm to ambient temperature and stirred overnight. The reaction was diluted with DCM (100 ml) and washed with 2M sodium hydroxide (2×50 ml) and then brine (50 ml), dried over magnesium sulfate, filtered and concentrated in vacuo to leave a residue which was purified by flash chromatography using a gradient elution of 0 to 50% ethyl acetate in iso-hexane to give a white solid (1.11 g).

NMR (CDCl₃): 0.97 (s, 3H), 1.35 (m, 4H), 1.45 (s, 9H), 1.80 (m, 4H), 2.11 (m, 2H), 2.85 (m, 4H), 3.00 (m, 1H), 3.17 (m, 2H), 3.63 (m, 2H), 4.37 (m, 2H), 5.14 (s, 2H), 7.28-7.41 (m, 5H); M+Na 531.

### Step 2: Preparation of title compound

To tert-butyl 4-[2-({1-[(benzyloxy)carbonyl]piperidin-4-yl}sulfonyl)ethyl]-4-methylpiperidine-1-carboxylate (1.11g, 2.19 mmol) was added a 4M solution of hydrochloric acid in dioxane (22 ml) and the mixture was stirred for 1 hour and then concentrated in vacuo. The residue was partitioned between DCM (50 ml) and 2M NaOH (50 ml) and the aqueous layer separated and washed with further DCM (50 ml). The organic layers were combined and dried over magnesium sulphate, filtered and concentrated in vacuo to give a yellow foam (1.04 g).

NMR (CDCl₃): 0.97 (s, 3H), 1.36 (m, 4H), 1.79 (m, 4H), 2.09 (m, 2H), 2.84 (m, 6H), 3.01 (m, 1H), 4.37 (m, 2H), 5.14 (s, 2H), 7.29-7.41 (m, 8H); M+H 409.

Method U

Preparation of 4-(2-{[4-(methylsulfonyl)phenyl]sulfonyl}ethyl)piperidine-4-carbonitrile

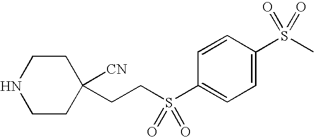

### Step 1: Preparation of tert-butyl 4-(2-{[tert-butyl(dimethyl)silyl]oxy}ethyl)-4-cyanopiperidine-1-carboxylate

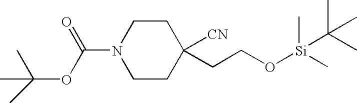

To a solution of tert-butyl 4-cyanopiperidine-1-carboxylate (8.0 g) in anhydrous tetrahydrofuran, cooled to −10° C., was added a solution of lithium hexamethyldisilazane (1M in tetrahydrofuran, 38 mL). Meanwhile a pressure-equalizing dropping funnel was charged with a solution of (2-bromoethoxy)-tert-butyldimethylsilane (8.16 mL) in tetrahydrofuran. This solution was added slowly to the cooled stirred reaction solution. Once addition was complete the reaction mixture was allowed to warm to room temperature and left to stir overnight. The reaction mixture was then quenched with a solution of saturated brine and extracted into ethyl acetate. The organic layer was separated and the aqueous portion was further extracted into ethyl acetate. The combined ethyl acetate extracts were washed with brine and dried over magnesium sulfate. Filtration and evaporation of solvents under reduced pressure gave the crude product which was purified by silica chromatography, eluting with a gradient of ethyl acetate in iso-hexane, to give the tert-butyl 4-(2-{[tert-butyl(dimethyl)silyl]oxy}ethyl)-4-cyanopiperidine-1-carboxylate (10.233 g) as a clear oil.

MS (ES) 313 (M-^{t}Bu)H⁺NMR (CDCl₃): −0.01 (s, 9H), 0.82 (s, 9H), 1.36-1.46 (m, 11H), 1.73 (t, 2H), 1.89 (d, 2H), 2.98 (t, 2H), 3.80 (t, 2H), 3.97-4.06 (m, 2H).

### Step 2: Preparation of tert-butyl 4-cyano-4-(2-hydroxyethyl)piperidine-1-carboxylate

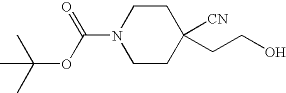

tert-Butyl 4-(2-{[tert-butyl(dimethyl)silyl]oxy}ethyl)-4-cyanopiperidine-1-carboxylate (7.000g) was dissolved in tetrahydrofuran and cooled to 0° C. To this was added tetra-N-butylammonium fluoride trihydrate (4.830g). The reaction was allowed to warm to room temperature and left to stir for 18 hours before being quenched by the addition of saturated ammonium chloride solution. The reaction was then extracted twice into ethyl acetate and the combined ethyl acetate portions were washed with brine, filtered and the solvents were removed under reduced pressure to give a clear oil. This was purified by silica chromatography, eluting with a gradient of ethyl acetate in iso-hexane, to give the tert-butyl 4-cyano-4-(2-hydroxyethyl)piperidine-1-carboxylate (4.160g) as a clear oil.

NMR (CDCl₃): 1.43-1.54 (m, 1H), 1.69 (s, 1H), 1.86 (t, 2H), 1.98 (d, 2H), 3.05 (t, 2H), 3.93 (t, 2H), 4.10 (s, 2H).

### Step 3: Preparation of 4-cyano-4-(2-{[4-(methylthio)phenyl]thio}ethyl)piperidine-1-carboxylate

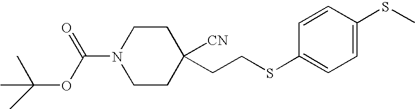

tert-Butyl 4-cyano-4-(2-hydroxyethyl)piperidine-1-carboxylate (3.480g) was dissolved in anhydrous dichloromethane and to this was added di-iso-propylethylamine. The reaction mixture was then cooled to −10° C. with stirring. Meanwhile a pressure-equalizing dropping funnel was charged with a solution of methanesulfonyl chloride (1.166 mL) in dichloromethane. The solution of methanesulfonyl chloride was then added slowly to the stirred reaction mixture, and once addition was complete the reaction mixture was warmed to room temperature and left to stir for 18 hours. The reaction was then quenched by the addition of saturated brine solution and extracted twice with dichloromethane. The combined dichloromethane extracts were then filtered under suction, and evaporation of solvents under reduced pressure gave tert-butyl 4-cyano-4-{2-[(methylsulfonyl)oxy]ethyl}piperidine-1-carboxylate as a fawn oil (4.420g). Meanwhile, anhydrous N,N-dimethylformamide was added to a portion of sodium hydride (60% dispersion in mineral oil, 0.685 g) and the resulting blue-grey suspension was cooled to 0° C. To this was slowly added 4-(methylthio)benzenethiol (2.68 g) then the reaction was left to stir at 0° C. for 20 minutes. To the stirred reaction mixture was then slowly added a solution of tert-butyl 4-cyano-4-{2-[(methylsulfonyl)oxy]ethyl}piperidine-1-carboxylate (2.680g) in anhydrous N,N-dimethylformamide. The reaction mixture was then allowed to warm to room temperature before being quenched by the addition of water and extracted twice into ethyl acetate. The combined ethyl acetate extracts were then washed with brine and dried over magnesium sulfate. Filtration of solvents under reduced pressure gave a yellow oil which was purified by silica chromatography, eluting with a gradient of ethyl acetate in iso-hexane, to give the tert-butyl 4-cyano-4-(2-{[4-(methylthio)phenyl]thio}ethyl)piperidine-1-carboxylate (3.410g) as a pale yellow gum.

MS (ES) 293 (M-Boc)H⁺

NMR (CDCl₃): 1.35-1.43 (m, 2H), 1.45 (s, 9H), 1.83-1.93 (m, 4H), 2.47 (s, 3H), 2.97-3.06 (m, 4H), 4.08-4.16 (m, 2H), 7.19 (d, 2H), 7.29 (d, 2H).

### Step 4: Preparation of title compound

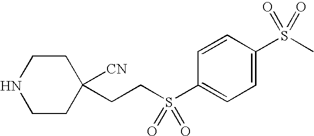

tert-Butyl 4-cyano-4-(2-{[4-(methylthio)phenyl]thio}ethyl)piperidine-1-carboxylate (1.700g) was dissolved in dichloromethane and cooled to 0° C. with stirring. To this was added meta-chloroperbenzoic acid (4.27 g at approx. 70% strength). Reaction was allowed to come to room temperature slowly, then left to stir for 18 hours before addition of aqueous 1N sodium hydroxide. The reaction was stirred for a further 30 minutes then was extracted with dichloromethane and the dichloromethane extract was then washed with brine, dried over magnesium sulfate and filtered under suction. Evaporation of the filtrate solution under reduced pressure gave a white solid which was then dissolved in 1,4-dioxane and stirred at room temperature. To this was then added a solution of HCl in 1,4-dioxane (4M, 50 mL) and the resulting white suspension was left to stir for 24 hours. The reaction mixture was filtered under reduced pressure then the filter cake was washed with diethyl ether and air-dried to give 4-(2-{[4-(methylsulfonyl)phenyl]sulfonyl}ethyl)piperidine-4-carbonitrile hydrochloride (1.499 g) as a white solid.

MS (ES) 357 (M+H)⁺

NMR (DMSO) δ: 1.83 (t, 2H), 1.99-2.04 (m, 2H), 2.17 (d, 2H), 2.87 (q, 2H), 3.31-3.38 (m, 5H), 3.60-3.65 (m, 2H), 8.24 (s, 4H), 9.24 (s, 2H).

Method V

### Preparation of (1S)-3-chloro-1-(3,5-difluorophenyl)propan-1-ol

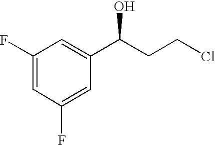

### Step 1: Preparation of 3-chloro-1-(3,5-difluorophenyl)propan-1-one

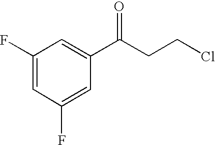

A mixture of 3-chloropropionyl chloride (4.77 ml, 50 mmol), manganese chloride (189 mg, 1.5 mmol), lithium chloride (127 mg, 3 mmol) and copper (I) chloride (149 mg, 1.5 mmol) was stirred in dry THF (50 ml) under an atmosphere of argon for 1 hour. The resulting mixture was cooled to 0° C. and 0.5M solution of 3,5-difluorophenyl magnesium bromide (100 ml, 50 mmol) was added via syringe pump over 1 hour. After the addition was complete the reaction was stirred for a further 10 minutes and then 1M HCl (50 ml) added. The mixture was extracted with diethyl ether (3×50 ml), washed with water (100 ml) then brine (100 ml), dried over magnesium sulphate, filtered and concentrated in vacuo. Purified by flash chromatography using a gradient elution of 0 to 10% ethyl acetate in iso-hexane to give an off-white solid (5.60 g).

NMR (CDCl₃): 3.41 (t, 2H), 3.92 (t, 2H), 7.05 (m, 1H), 7.47 (m, 2H).

### Step 2: Preparation of title compound

(R)-2-Diphenyl-2-pyrrolidinemethanol (694 mg, 2.74 mmol) was dissolved in dry THF under an atmosphere of argon and trimethyl borate (369 μl, 0.12 mmol) added. The reaction was stirred for 2 hours and then borane.dimethylsulfide complex (2.60 ml, 27.4 mmol) was added. The mixture was cooled to −4° C. and 3-chloro-1-(3,5-difluorophenyl)propan-1-one (5.60 g, 27.4 mmol) in dry THF (70 ml) was added via syringe pump over 1 hour. The reaction was allowed to warm to ambient temperature and then stirred overnight, cooled to 0° C. and methanol (30 ml) added followed by a solution of 4M HCl in dioxane (7 ml) and the mixture concentrated in vacuo. Toluene (40 ml) was added and the white solid filtered off and the filtrate concentrated in vacuo to give a yellow oil (5.47 g).

NMR (CDCl₃): 2.12 (m, 2H), 3.58 (m, 1H), 3.75 (m, 1H), 4.96 (m, 1H), 6.73 (m, 1H), 6.92 (m, 2H).

### EXAMPLE 4

The ability of compounds to inhibit the binding of RANTES was assessed by an in vitro radioligand binding assay. Membranes were prepared from Chinese hamster ovary cells which expressed the recombinant human CCR5 receptor. These membranes were incubated with 0.1 nM iodinated RANTES, scintillation proximity beads and various concentrations of the compounds of the invention in 96-well plates. The amount of iodinated RANTES bound to the receptor was determined by scintillation counting. Competition curves were obtained for compounds and the concentration of compound which displaced 50% of bound iodinated RANTES was calculated (IC₅₀). Certain compounds of the invention have an IC₅₀ of less than 50 μM.

### EXAMPLE 5

The ability of compounds to inhibit the binding of MIP-1α was assessed by an in vitro radioligand binding assay. Membranes were prepared from Chinese hamster ovary cells which expressed the recombinant human CCR5 receptor. These membranes were incubated with 0.1 nM iodinated MIP-1α, scintillation proximity beads and various concentrations of the compounds of the invention in 96-well plates. The amount of iodinated MIP-1α bound to the receptor was determined by scintillation counting. Competition curves were obtained for compounds and the concentration of compound which displaced 50% of bound iodinated MIP-1α was calculated (IC₅₀). Certain compounds of the invention have an IC₅₀ of less than 50 μM.

Results from this test for certain compounds of the invention are presented in Table VI. In Table X the results are presented as Pic50 values. A Pic50 value is the negative log (to base 10) of the IC₅₀ result, so an IC50 of 1 μM (that is 1×10⁻⁶M) gives a Pic50 of 6. If a compound was tested more than once then the data below is an average of the probative tests results.

TABLE X

| Table Number | Compound number | Pic50 |
|---|---|---|
| I | 1 | 9.6 |
| I | 4 | 8.9 |
| I | 5 | 8.0 |
| I | 6 | 8.5 |
| II | 1 | 9.2 |
| II | 2 | 9.3 |
| II | 4 | 9.4 |
| II | 11 | 9.5 |
| II | 12 | 8.3 |
| II | 13 | 9.3 |
| II | 14 | 9.4 |
| II | 15 | 9.5 |
| II | 16 | 9.3 |
| II | 17 | 9.5 |
| II | 18 | 9.2 |
| II | 19 | 8.7 |
| II | 20 | 8.3 |
| II | 21 | 7.7 |
| III | 1 | 9.5 |
| V | 1 | 5.8 |
| V | 2 | 8.0 |
| VI | 1 | 8.3 |
| VII | 1 | 6.3 |
| VII | 2 | 8.1 |
| VIII | 1 | 8.1 |
| VIII | 2 | 8.7 |
| VIII | 3 | 8.3 |
| IX | 1 | 7.3 |

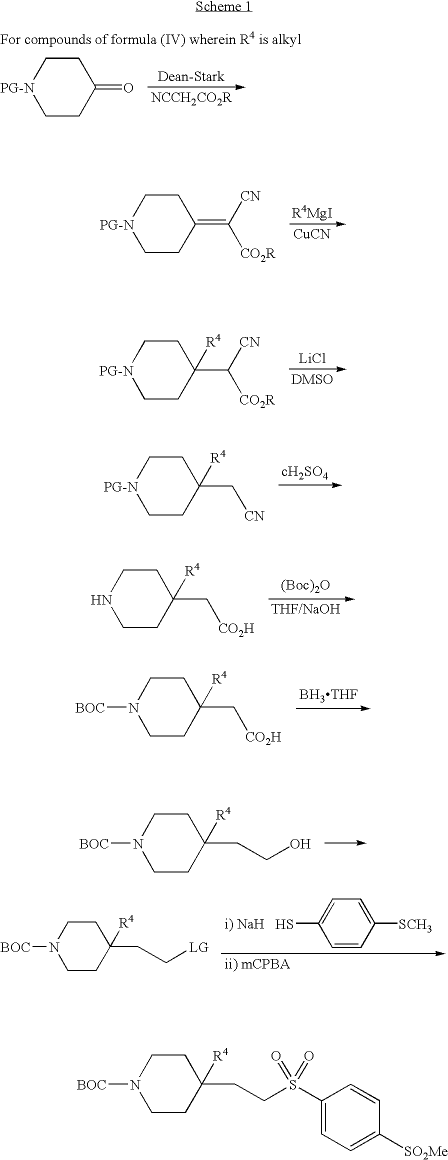

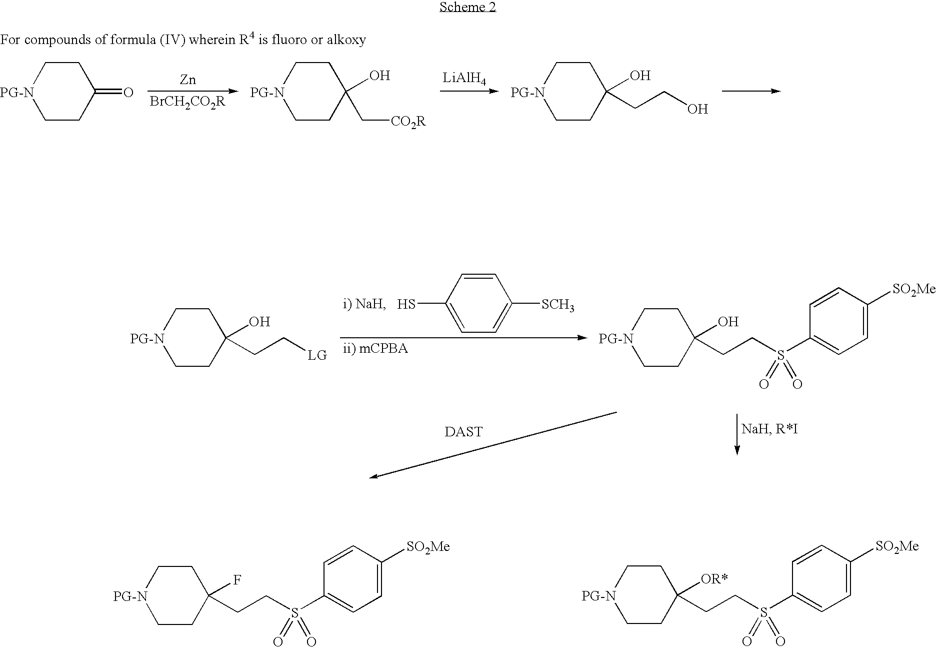

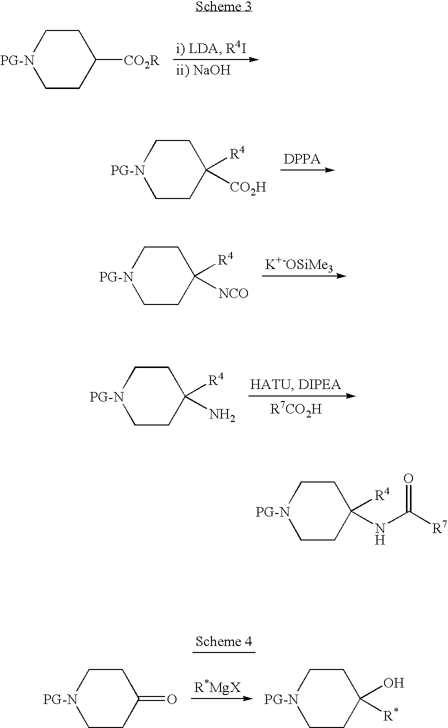

1. A compound of formula (I): 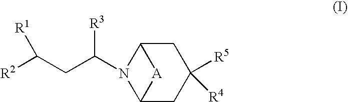
wherein:
A is absent or it is CH₂CH₂;
R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, heterocyclyl, aryl or heteroaryl;
R¹⁴, R¹⁷, R¹⁹, R²⁰ and R²² are hydrogen or C₁₋₆ alkyl;
R¹⁵, R¹⁶, R¹⁸, R²¹ and R²³ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₚ(C₁₋₆ alkyl), halo or C₁₋₄ alkyl); or R¹⁵, R¹⁶, R¹⁸ and R²¹ can also be hydrogen;
or R¹⁴ and R¹⁵, and/or R²⁰ and R²¹ may join to form a 4-, 5- or 6-membered ring which optionally includes a nitrogen, oxygen or sulphur atom, said ring being optionally substituted by halo, C₁₋₆ alkyl, S(O)₁(C₁₋₆ alkyl) or C(O)(C₁₋₆ alkyl);
R² is phenyl or heteroaryl, either of which is optionally substituted by halo, C₁₋₄ alkyl, C₁₋₄ alkoxy, cyano or CF₃;
R³ is hydrogen or C₁₋₄ alkyl;
R⁴ is halo, hydroxy, cyano, C₁₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);
R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl (optionally substituted by halogen, C₁₋₄ alkoxy or OH), H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring;
the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro};
X is O, S(O)ₚ, S(O)₂NR⁸ or NR⁸S(O)₂;
m and n are 1, 2 or 3;
R⁶ is hydrogen, methyl, ethyl, allyl or cyclopropyl;
R⁷ is phenyl, heteroaryl, phenylNR¹¹, heteroarylNR¹¹, phenyl(C₁₋₂)alkyl, heteroaryl(C₁₋₂)alkyl, phenyl(C₁₋₂ alkyl)NH or heteroaryl(C₁₋₂ alkyl)NH; wherein the phenyl and heteroaryl rings of R⁷ are optionally substituted by halo, cyano, nitro, hydroxy, C₁₋₄ alkyl, C₁₋₄ alkoxy, S(O)ₖ(C₁₋₄ alkyl), S(O)₂NR¹²R¹³, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁸ and R¹¹ are, independently, hydrogen, C₁₋₆ alkyl or C₃₋₇ cycloalkyl;
R⁹ is aryl, heteroaryl, C₁₋₆ alkyl, C₃₋₇ cycloalkyl or heterocyclyl;
R¹⁰ aryl, heteroaryl or heterocyclyl;
R¹² and R¹³ are, independently, hydrogen or C₁₋₄ alkyl, or together with a nitrogen or oxygen atom, may join to form a 5- or 6-membered ring which is optionally substituted with C₁₋₄ alkyl, C(O)H, C(O)(C₁₋₄ alkyl) or SO₂(C₁₋₄ alkyl);
aryl, phenyl and heteroaryl moieties are independently optionally substituted by one or more of halo, cyano, nitro, hydroxy, OC(O)NR²⁴R²⁵, NR²⁶R²⁷, NR²⁸C(O)R²⁹, NR³⁰C(O)NR³¹R³², S(O)₂NR³³R³⁴, NR³⁵S(O)₂R³⁶, C(O)NR³⁷R³⁸, CO₂R³⁹, NR⁴¹CO₂R⁴¹, S(O)_{q}R⁴², OS(O)₂R⁴³, C₁₋₆ alkyl (optionally mono-substituted by S(O)₂R⁴⁴ or C(O)NR⁴⁵R⁴⁶), C₂₋₆ alkenyl, C₂₋₆ alkynyl, C₃₋₁₀cycloalkyl, C₁₋₆ haloalkyl, C₁₋₆ alkoxy(C₁₋₆)alkyl, C₁₋₆ alkoxy (optionally mono-substituted by CO₂R⁴⁷, C(O)NR⁴⁸R⁴⁹, cyano, heteroaryl or C(O)NHS(O)₂R⁵⁰), NHC(O)NHR⁵¹, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(Cf 4)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃;
unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁵²R⁵³, C(O)R⁵⁴, C(O)₂(C₁₋₆ alkyl) (such as tert-butoxycarbonyl), C(O)₂(phenyl(C₁₋₂ alkyl)) (such as benzyloxycarbonyl), C(O)NHR⁵⁵, S(O)₂R⁵⁶, NHS(O)₂NHR⁵⁷, NHC(O)R⁵⁸, NHC(O)NHR⁵⁹ or NHS(O)₂R⁶⁰, provided none of these last four substituents is linked to a ring nitrogen;
k, l, p and q are, independently, 0, 1 or 2;
R²⁴, R²⁶, R²⁸, R³⁰, R³¹, R³³, R³⁵, R³⁷, R⁴⁰, R⁵², R⁴⁵ and R⁴⁸ are, independently, hydrogen or C₁₋₆ alkyl;
R²⁵, R²⁷, R²⁹, R³⁴, R³⁶, R³⁸, R³⁹, R⁴¹, R⁴², R⁵³, R⁵⁴, R⁵⁵, R⁵⁶, R⁵⁷, R⁵⁸, R⁵⁹, R⁶⁰, R⁴³, R⁴⁴, R⁴⁶, R⁴⁷, R⁴⁹, R⁵⁰ and R⁵¹ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
R²⁵, R²⁷R²⁹R³²R³⁴R³⁸R³⁹R⁵³R⁵⁴, R⁵⁵, R⁵⁷, R⁵⁸, R⁵⁹, R⁴⁶, R⁴⁷, R⁴⁹ and R⁵¹ may additionally be hydrogen;
or a pharmaceutically acceptable salt thereof;
provided that when R¹ is an optionally substituted isolated 6-membered heterocyclyl and R⁴ is C₁₋₃ alkyl, then R⁵ is not an optionally substituted five membered heterocycle containing at least one carbon atom, one to four nitrogen atoms and, optionally, one oxygen or sulphur atom, said five membered heterocycle being optionally fused to another ring.

2. A compound of formula (I) as claimed in claim 1 wherein:
R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, aryl or heteroaryl;
R⁴ is halo, hydroxy, cyano, C₁₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);
R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl, H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring; the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro};
R², R³, A, X, m, n, R⁶, R⁷, R⁹, R10, R¹⁴, R¹⁵, R¹⁶, R¹⁷, R¹⁸, R¹⁹, R²⁰, R²¹, R²², R²³ are as defined in claim 1; and,
aryl and heteroaryl moieties are independently optionally substituted as recited in claim 1;
or a pharmaceutically acceptable salt thereof.

3. A compound of formula (I) as claimed in claim 1 wherein:
R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, heterocyclyl, aryl or heteroaryl;
R⁴ is halo, hydroxy, cyano, C₁₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(C₁₋₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);
R⁵ is aryl, (CH₂)ₙXR⁹ or (CH₂)ₘR^{l0}, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷;
R², R³, A, X, m, n, R⁶, R⁷, R⁹, R10, R¹⁴, R¹⁵, R¹⁶, R¹⁷, R¹⁸, R¹⁹, R²⁰, R²¹, R²² and R²³ are as defined in claim 1; and,
heterocyclyl, aryl and heteroaryl moieties are independently optionally substituted as recited in claim 1;
or a pharmaceutically acceptable salt thereof.

4. A compound of formula (I) as claimed in claim 1 wherein:
R¹ is C₁₋₈ alkyl, C(O)NR¹⁴R¹⁵, C(O)₂R¹⁶, NR¹⁷C(O)R¹⁸, NR¹⁹C(O)NR²⁰R²¹, NR²²C(O)₂R²³, heterocyclyl, aryl or heteroaryl;
R⁴ is halo, hydroxy, cyano, C₄₋₆ alkyl, CF₃, OCF₃, C₁₋₄ alkoxy(C₁₋₆)alkyl, C₁₋₆ alkoxy, C(O)NH₂, C(O)NH(C₁₋₄ alkyl), C(O)N(C₁₋₄ alkyl)₂, NH₂, NH(C₁₋₄ alkyl), N(Cl₄ alkyl)₂, C(O)(C₁₋₄ alkyl), S(O)₂(C₁₋₄ alkyl), N(C₁₋₄ alkyl)C(O)C₁₋₄ alkyl, N(C₁₋₄ alkyl)S(O)₂(C₁₋₄ alkyl) or N(C₁₋₄ alkyl)C(O)O(C₁₋₄ alkyl);
R⁵ is aryl, (CH₂)ₙXR⁹ or (CH₂)ₘR¹⁰, or, when R⁴ is alkyl, CF₃, alkoxy(C₁₋₆)alkyl, C(O)NH₂, C(O)NH(C₁₋₄ alkyl) and C(O)N(C₁₋₄ alkyl)₂, then R⁵ can also be NR⁶C(O)R⁷, or a five membered heterocycle containing at least one carbon atom, one to four nitrogen atoms and, optionally, one oxygen or sulphur atom, said heterocycle being optionally substituted by oxo, C₁₋₆ alkyl, H₂NC(O), (phenylC₁₋₂ alkyl)HNC(O) or benzyl [which is optionally substituted by halogen, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl)]; the five membered heterocycle being optionally fused to a cyclohexane, piperidine, benzene, pyridine, pyridazine, pyrimidine or pyrazine ring; the ring carbon atoms of said fused cyclohexane, piperidine, benzene, pyridine, pyridazine, pyrimidine or pyrazine ring being optionally substituted by halogen, cyano, C₁₋₄ alkyl, C₁₋₄ alkoxy, CF₃, OCF₃, S(C₁₋₄ alkyl), S(O)(C₁₋₄ alkyl) or S(O)₂(C₁₋₄ alkyl); and the nitrogen of the fused piperidine ring being optionally substituted by C₁₋₄ alkyl {which is optionally substituted by oxo, halogen, OH, C₁₋₄ alkoxy, OCF₃, C(O)O(C₁₋₄ alkyl), CN, C(O)NH₂, C(O)NH(C₁₋₄ alkyl), C(O)N(C₁₋₄ alkyl)₂, NH₂, NH(C₁₋₄ alkyl) or N(C₁₋₄ alkyl)₂}, C(O)(C₁₋₄ alkyl) {wherein the alkyl is optionally substituted by C₁₋₄ alkoxy or fluoro}, C(O)O(C₁₋₄ alkyl), C(O)NH₂, C(O)NH(C₁₋₄ alkyl), C(O)N(C₁₋₄ alkyl)₂ or S(O)₂(C₁₋₄ alkyl) {wherein the alkyl is optionally substituted by fluoro};
R², R³,A, X, m, n, R⁶, R⁷, R⁹, R10, R¹⁴, R¹⁵, R¹⁶, R¹⁷, R¹⁹, R²⁰,R²¹, R²² and R²³ are as defined in claim 1; and,
heterocyclyl, aryl and heteroaryl moieties are independently optionally substituted as recited in claim 1;
or a pharmaceutically acceptable salt thereof.

5. A compound as claimed in claim 1 wherein R¹ is:
1-substituted piperidin-4-yl or a 4-substituted piperazin-1-yl, wherein the substituent is S(O)₂(C₁₋₄ alkyl), S(O)₂(C₁₋₄ haloalkyl), S(O)₂(phenyl), S(O)₂N(C₁₋₄ alkyl)₂ or phenyl;
NHC(O)R¹⁸ wherein R¹⁸ is C₁₋₄ haloalkyl, phenyl (optionally substituted by halo) or
C₃₋₆ cycloalkyl (substituted by one or two fluoros);
phenyl optionally substituted by S(O)₂R⁴² (wherein R⁴² is C₁₋₄ alkyl); or,
heterocyclyl.

6. A compound as claimed in any one of the preceding claims wherein R² is phenyl;
phenyl substituted by halo and/or CF₃; or thienyl substituted by halo.

7. A compound as claimed in any one of the preceding claims wherein R³ is hydrogen.

8. A compound as claimed in any one of the preceding claims wherein A is absent.

9. A compound as claimed in any one of the preceding claims wherein R⁴ is halo, hydroxy, C₁₋₆ alkyl or C₁₋₆ alkoxy.

10. A compound as claimed in any one of the preceding claims wherein R⁵ is CH₂CH₂S(O)R⁹; wherein R⁹ is as defined in claim 1.

11. A compound as claimed in any one of the preceding claims wherein R¹⁵ is NR⁵⁶C(O)R⁷; wherein R⁶ and R⁷ are as defined in claim 1.

12. A process for preparing of a compound as claimed in claim 1, the process comprising:
a. reductive amination of a compound of formula (II): 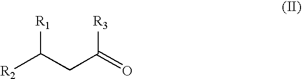
wherein R¹, R² and R³ are as defined above, with a compound of formula (III): 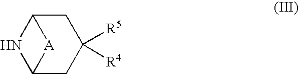
wherein R⁴, R⁵ and A are as defined above, in the presence of NaBH(OAc)₃ in a suitable solvent at room temperature; or,
b. alkylation of a compound of formula (III) with a compound of formula (V): 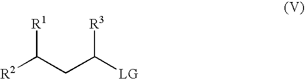
wherein R¹, R² and R³ are as defined above and LG is a leaving group, in the presence of a suitable base, in a suitable solvent, at a suitable temperature.

13. A pharmaceutical composition which comprises a compound as claimed in claim 1, or a pharmaceutically acceptable salt thereof, and a pharmaceutically acceptable adjuvant, diluent or carrier.

14. A compound as claimed in claim 1, or a pharmaceutically acceptable salt thereof, for use as a medicament.

15. A compound as claimed in claim 1, or a pharmaceutically acceptable salt thereof, in the manufacture of a medicament for use in therapy.

16. A method of treating a CCR5 mediated disease state comprising administering to a patient in need of such treatment an effective amount of a compound as claimed in claim 1, or a pharmaceutically acceptable salt thereof.
